(19) United States
(12) Patent Application Publication
(10) Pub. No.: US 2023/0263482 A1
(43) Pub. Date: Aug. 24, 2023

(54) SYSTEMS, METHOD, AND APPARATUS FOR PROVIDING PERSONALIZED MEDICAL DATA

(71) Applicant: Johnson & Johnson Consumer Inc., Skillman, NJ (US)

(72) Inventors: Stefan Kostense, Voorburg (NL); Kevin J. Wildenhaus, Plymouth, MI (US)

(21) Appl. No.: 18/111,121

(22) Filed: Feb. 17, 2023

Related U.S. Application Data
(60) Provisional application No. 63/313,462, filed on Feb. 24, 2022.

(51) Int. Cl. A61B 5/00; G16H 10/60; G16H 10/20; G16H 50/20

(52) U.S. Cl. CPC A61B 5/744; A61B 5/748; A61B 5/7275; G16H 10/60; G16H 10/20; G16H 50/20

(57) ABSTRACT

A device disclosed herein may be used for providing personal medical data. The device may comprise a memory and/or a processor. The processor may be configured to perform a number of actions. A graphic of a human body may be displayed. A user input associated with a location on the graphic of a human body may be received from a user. An organ context may be determined based on the location on the graphic of the human body. A biomarker related to the organ context may be determined. Contextualized health data that indicates a significance of the biomarker in relation to the organ context may be generated. In response to the user input, the device may display the contextualized health data, a recommended action, and an indication of an amount of time that the user's life may be extended by the user performing the recommended action.

### CROSS-REFERENCE TO RELATED APPLICATION

This application claims the benefit of U.S. Provisional Patent Application No. 63/313,462 filed Feb. 24, 2022, the contents of which are hereby incorporated herein by reference.

### BACKGROUND

Medical information and/or medical statuses may be difficult and boring for users to read when presented in tables and lists. Users may be more engaged with their personal data and medical status if the interaction is more personalized.

### SUMMARY

An interface between personalized medical data and the user may be provided. The interface may provide a graphic of a human body that may be personalized into a personal avatar. For example, a user may tap on different body parts of the avatar to render the data/information that may be relevant to that body part. Tapping the chest area may visualize the heart, and another tap may show the status of one or more heart measurements such as a current heart rate, a heart rate trend, a comparison to normal/healthy heart rate range, and/or the like. Users may further click to get tips, suggestions, and techniques on health related to a body part.

A device disclosed herein may be used for providing personal medical data. The device may comprise a memory and/or a processor. The processor may be configured to perform one or more actions. A graphic of a human body may be displayed. A user input associated with a location on the graphic of a human body may be received from a user. An organ context may be determined based on the location on the graphic of the human body. A biomarker related to the organ context may be determined. Contextualized health data that indicates a significance of the biomarker in relation to the organ context may be generated. In response to the user input, the device may display the contextualized health data, a recommended action, and an indication of an amount of time that the user's life may be extended by the user performing the recommended action.

An organ context may be determined. A biomarker related to the organ context may be determined. The contextualized health data for the organ context may be determined and/or generated. The contextualized health data may indicate a significance of the biomarker. A recommended action (e.g., a preventative measure) may be determined and/or displayed. The recommended action may indicate an action that a user may perform to improve a health issue related to the organ context.

A device disclosed herein may be used for providing a personalized medical data notification. The device may comprise a memory and/or a processor. The processor may be configured to perform one or more actions. A biomarker may be determined for a user. The biomarker may indicate a health issue related to an organ context. A notification may be displayed to the user. The notification may indicate contextualized data for the user that may include the biomarker, the organ context, and the health issue.

### BRIEF DESCRIPTION OF THE DRAWINGS

FIG. 1 depicts an example functional block diagram of certain electrical components of an example smart device for providing personalized medical data.

FIG. 2A depicts an example architecture diagram for an example system to support a smart device; FIG. 2B is a messaging flow diagram for the example system.

FIG. 3 depicts a block diagram of an example device that may include one or more modules (e.g., software modules) for providing personalized medical data, statuses, and/or recommendations.

FIG. 4 depicts an example method for providing personalized medical data, statuses, and/or recommendations.

FIG. 5 depicts an example method for using an organ context and/or a biomarker to provide personalized medical data, statuses, and/or recommendations.

FIG. 6 depicts an example method for using an organ context and/or a contextual health data to provide a personalized medical data notification.

FIG. 7 depicts an example block diagram of an example system that may include one or more devices to provide a customized health recommendation.

FIG. 8 depicts an example user interface that may include a customizable avatar for providing personalized medical data.

FIG. 9A-B depict example user interfaces for providing personalized medical data, statuses, and/or recommendations.

FIG. 10 depicts an example method for providing personalized medical data, statuses, and/or recommendations using risk assessments and/or risk analysis.

FIG. 11A-B depicts example user interfaces for providing personalized medical data, statuses, and/or recommendations using risk assessments and/or risk analysis.

### DETAILED DESCRIPTION

An example interface between personalized medical data and the user is provided herein. The interface may provide (e.g., display) a graphic of a human body that may be personalized into a personal avatar. For example, a user may tap on different body parts of the avatar to render the data/information that may be relevant to that body part. Tapping the chest area may visualize the heart, and another tap may show the status of one or more heart measurements such as a current heart rate, a heart rate trend, a comparison to normal/healthy heart rate range, and/or the like. Users may further click to get tips, suggestions, and techniques on health related to a body part.

A device disclosed herein may be used for providing personal medical data. The device may comprise a memory and/or a processor. The processor may be configured to perform one or more actions. A graphic of a human body may be displayed. A user input may be received (e.g., from a user), the user input being associated with a location on the graphic of a human body. An organ context may be determined based on the location on the graphic of the human body. A biomarker related to the organ context may be determined. Contextualized health data may be generated. The contextualized health data may indicate a significance of the biomarker in relation to the organ context. In response to the user input, the device may display the contextualized health data, a recommended action, and an indication of an amount of time that the user's life may be extended by the user performing the recommended action.

An organ context may be determined. A biomarker related to the organ context may be determined. The contextualized health data for the organ context may be determined and/or generated. The contextualized health data may indicate a significance of the biomarker. A recommended action (e.g., preventative measure) may be determined and/or displayed. The recommended action may indicate an action that a user may perform to improve a health issue related to the organ context.

Examples provided herein describe biomarkers that may be used to help identify people at risk for certain diseases. When certain biomarkers are determined, examples herein provide ways of using them. Biomarker information (e.g., all biomarker information) may be collected in an application. For example, biomarker information may be captured, measured, gathered, received, and/or determined by an application. In an example, the application may determine and/or receive biomarker information from a database, a server, a sensor, a medical device, an electronic medical record, a wearable device, a smart phone, a smart watch, and/or the like. The application may help engage users of the application and may help the users stay interested in the details (e.g., scientific details) that may be provided. The application may be presented in a way that is understandable to lay users (e.g., like a game).

The application may allow users to personalize a certain figure of a body that is the user's body, e.g., such as a digital avatar. Users may tap on whatever part of the body they want to know more about. After the body part is tapped, the application may provide biomarker information regarding that body part. In examples, if a user has a stomachache, they may select (e.g., push on) the stomach of the avatar. The stomach biomarkers may then pop up and tell the user they have been drinking too much alcohol, for example. As such, the application may be more interesting for people that do not know much about biomarkers. For example, the application may indicate to a user how the user's behaviors and/or diseases may interact between two or more organ systems. For example, poor diet may exacerbate stomach issues, may increase blood pressure, and may affect the heart.

Various technologies may be used to sense, track, and/or capture healthcare data. Certain biomarker tests may assemble and collate the healthcare data and then provide the information (e.g., via a personal dashboard) to the user, such that the user may receive a health readout on a regular (e.g., daily) basis with real-time notifications on specific health issues that may emerge. The notifications may allow users to better manage their health and ideally prevent more serious health issues (e.g., low blood sugar, a cardiac event, etc.). The application may be able to identify body parts in a gamification mechanism as a way to get people in touch with their health. Users may be able to monitor their heart rate, heart rate variability, blood pressure, carbon monoxide levels, breath diagnostics, measures of lung capacity, etc. in real time. The application may provide the different points of information in an engaging, instructive manner. Rather than presenting information as a black and white series of numbers and ranges, the application may make the body parts color-coded and visual, making users more likely to read and engage with their information, to remember the information, find the information valuable, and actually utilize the information.

The application may provide prediction assessments when looking at demographics and other information, incorporating some biomarker data, etc. Personalized recommendations may be provided for people, such as provided suggestions of what to do and what not to do. The recommendations may entice users and help them understand how following the recommendations may have health benefits. In examples, users may be provided estimates of how many days of life may be added by quitting smoking today, by taking a daily aspirin, etc. The application may integrate one organ system with another. In examples, if a user is a smoker, and the user's lung health was a focus, biomarkers of lung cancer risk may be combined with other biomarkers and behavioral indices, which may provide information to the user that is related to lung cancer (as well as other health risks). Therefore, users would have a more engaging way of taking charge of their health.

In examples, if users have pain somewhere in the body, they may tap on the body part where they feel the pain and then biomarker data may pop up. In examples, the system may be able to alert the user if it detects biomarker values outside of an expected range. For example, the system may determine that a value of the biomarker is outside of an acceptable range of values, and display (e.g., in a location associated with an organ context) a notification indicating for the user to review the biomarker. The user may provide a user input by selecting the notification. If the system alerts the user, the body part of the affected organ may be emphasized (e.g., such as being lit up). The system may allow a user to better understand and/or manage their health by providing a source (e.g., a centralized source) for a user's medical data. For example, the system may provide a centralized source that may include one or more medical records and/or biomarker information.

The application may have access to the user's medical records. The medical records may be pre-loaded into the application. If a user has a history of certain health issues, the medical history of the user may be used by the application to analyze the diagnosis of the user. As such, the medical history of the user and measured biomarkers may give a context to what medical issues or potential medical issues may arise for the user.

In examples, a user may wear a compression sock that people at risk for diabetes would wear. In the compression sock, there may be a biomarker sensor that determines heat and pressure. A user may use the digital interface of the application to pair the digital interface with the biomarker sensor to help detect diabetes and blood clots in the leg. The device may (e.g., may also) provide a system that may have a framework adapted for specific conditions, general organ challenges, or specific devices and technologies as they emerge (e.g., conditions such as those related to blood clots and issues with the lung and the heart).

Over time, the application may receive more data, allowing it to become smarter as the data set gets larger. This may allow for better integration of conditions. In examples, if detecting lung cancer risk for smokers (e.g., via breath sensors and genetic testing), the heart health, risk of stroke, and hypertension may (e.g., may also) be considered along with the lung cancer risk or diagnosis.

The application may present healthcare data in a specific way that is more actionable for users. The healthcare data may be filtered to make it relevant to the user based on their selections and understanding of the context they are looking at. The application may use the user selection to make sense of the data itself. For example, as the application collects the information, the healthcare data may be interpreted differently depending on whether the user clicks on the brain or the foot.

The application may explain data back to a user. User interaction with the data may be actionable through color coding and simplistic approaches. For example, if a user has a headache and they tap on their brain, but their issue is head pressure, the application may describe blood pressure and the impact on headache. As an example, color may be used to describe moving from an elevated blood pressure, which may be red, to a first pressure level, which may be purple, and to a second pressure level, which may be blue. The color may indicate a visual representation of blood pressure, which a patient may not be able to see and/or feel. If instead the user's issue is that they are taking their blood pressure reading, and they are concerned with their blood pressure number, the application may describe managing their hypertension or their diabetes. The application may output different recommendations based on different content that may emerge and whether a user is concerned with a headache or with high blood pressure, for example, even if the data is the same.

The application may perform types of screening or risk assessment that may be quantitative in nature and/or may be psychometric in nature such that it makes specific recommendations to improve health or manage pain, for example. The application may function as a personal digital assistant (PDA) or smart device that captures information in real time. For example, if information is captured during the day before a user goes to sleep, when the user wakes up in the morning, they may observe a sound quality sleep of 6.8 hours overnight, for example. The sound quality sleep may be compared to the day before, week before, etc. For example, if a user is mildly dehydrated, the application may encourage the user to drink more water and reduce morning caffeine consumption. The application may refer the user to a doctor to get an in-depth diagnosis if the application detects a problem (e.g., while comparing the biomarker values received to the expected biomarker values).

The application may (e.g., may also) serve as a notification alert system (e.g., via a “check engine” light). For example, if a biomarker or sensor is abnormal, or other source(s) of data that are abnormal, a body part (e.g., on the avatar) where the abnormal data is occurring may light up like an icon alert. The icon alert may tell the user to pay attention to the abnormalities now, as well as provide a self-generated exploration about the user's health, body parts, and/or well-being.

The application may provide an educational informational approach to users (e.g., such as for managing diabetes, managing fibromyalgia, or managing general health). Tips, ideas, and suggestions may be provided to users. The tips, ideas, and suggestions may be medically approved and recommended (e.g., such as drinking 64 ounces of water every day, etc.). The application may provide a condition to monitor (e.g., such as oxygen rates for lung disease, blood pressure respiration rate, heart rate variability, inflammation for cardiovascular disease, etc.) and a tangible action to take associated with the output. This may help users get more specific and focal with the treatment when talking with their doctors and managing their symptoms.

The application may help users self-manage their symptoms, such as making recommendations and suggestions to help the user manage the headache or pain, improve their energy level, etc. Through interaction and capturing data via the application, users may self-manage and self-treat some of their milder symptoms. For example, a user with tension headaches may try progressive muscle relaxation work or try meditation to help them. Users may try both of those approaches for a series of time (e.g., three or four days) and determine which one works for them, enter information (e.g., input) into the data, and become self-managers of their condition.

The application may help users self-report information and help identify what their triggers and potential solutions may be. For example, the application may ask a user suffering with digestion issues what they ate and when they started feeling bad, their stress level, or other potential questions related to triggers of digestion issues. The application may start to capture information that may be used on a larger scale to compile data for several people struggling with digestion issues. The information may (e.g., may also) be used at the individual level to help users identify what their triggers are and then potential solutions that may treat their digestive issues. The user may start this process by touching the stomach of the digital body of the application (e.g., their digital avatar).

In examples, baseline biomarkers of inflammation may be calculated and may indicate that the user is predisposed to heart disease and/or at risk for heart attack. The application may provide the user with repeated measures to address the indicated issues (e.g., the user may change their diet, start using a probiotic, start using a highly concentrated fish oil supplement, etc.). The inflammation levels of the user may be monitored over time. The user may be able to see how the inflammation levels change (e.g., come down). A simple colored system (e.g., a red, yellow green system) may be used to indicate the inflammation levels, rather than black and white numbers on a page. In examples, low heart rate variability may be predictive of poor health. The application may demonstrate ways to increase heart rate variability and to measure over time how the user's heart rate variability changes and how their heart rate variability numbers compare to other people across similar demographics. This may allow user to detect individual changes over time. Users may map those changes compared to other people with similar ages and health issues, for example.

The application may show the organs of the digital avatar that relate to the condition or the issue that the user is concerned about. In examples, when a user clicks on the brain, the application may detect the specific issue the user is looking for related to the brain. If a specific issue is detected on the application, the application may notify the user of certain activities to perform (e.g., for the day) related to the brain issue for the user. In examples, the user may look for more general information regarding their brain issue. This may provide education to users who are using the application and may help them understand that when they click on different organs or body parts, the application may provide information relevant to their health issue.

Various sensors may provide one form of data input that may involve biomarkers, such as the biomarkers described herein. Some sensors may be worn by users consistently (e.g., day after day that may always be capturing information). Other sensors may be used periodically. Temporary-type sensors may capture data. Other forms of data inputs to measure biomarkers may be diagnostic or device-oriented (e.g., saliva samples and blood samples).

Other data capturing devices may provide a steppingstone toward capturing more specific types of data. For example, a user may be wearing a watch that captures information that suggests the person needs to wear a halter patch. The halter patch may identify that there is something related to pulmonary function that may lead the user to want to take a blood test. As such, data capturing may be used in stepwise fashion that may help to make decisions about deepening the screening or the testing process based on the data captured to better know when to do a blood test, for example. This may capture the baseline information that informs users whether they need to analyze health issues more in depth.

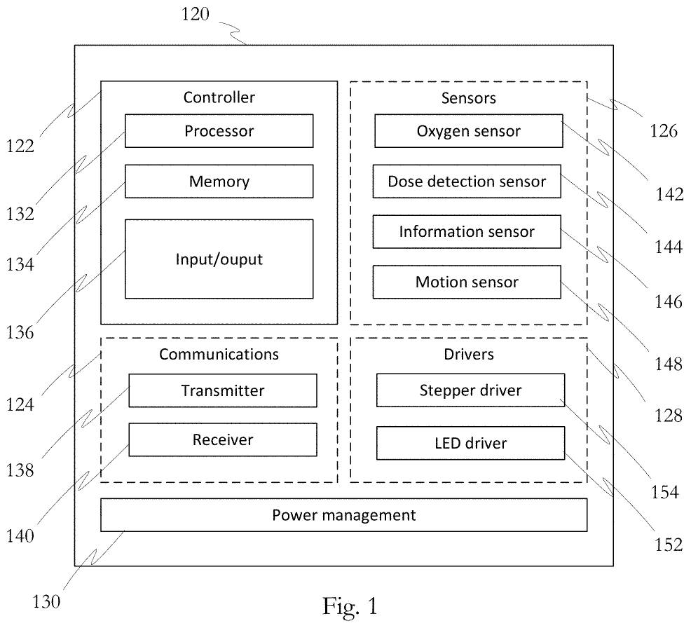

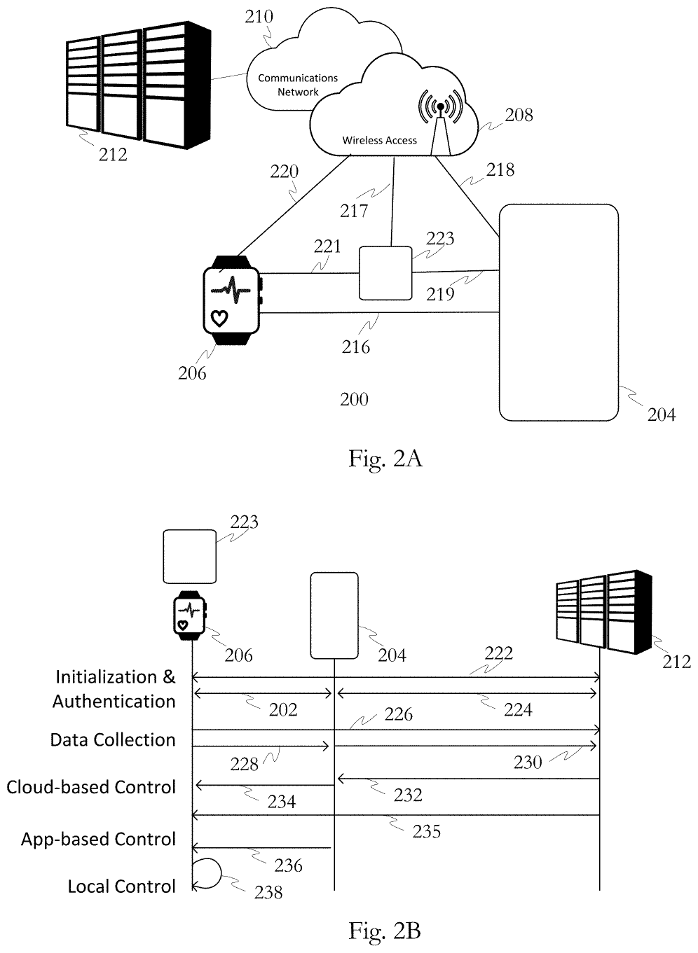

FIG. 1 depicts an example functional block diagram of certain electrical components of an example smart device for providing personalized medical data. The smart device may be a smart phone, a smart watch, a wearable device, a cellular phone, a computer, a servicer, and/or the like. These components 120 may be incorporated into the smart device, such as devices 206, 223, 204 (shown with respect to FIG. 2), and/or may be incorporated into a computing resource, such as 212 (also shown with respect to FIG. 2). Referring again to FIG. 1, the components 120 may integrate sensing, electromechanical driving, communications, and digital-processing functionality to the structure and operation of the dispenser. In examples, the components 120 may include a controller 122, communications interfaces 124, sensors 126, electrical and electromechanical drivers 128, and a power management subsystem 130.

The controller 122, may include a processor 132, a memory 134, and one or more input/output devices 136, for example. The controller 122 may be any suitable microcontroller, microprocessor, field programmable gate array (FPGA), application specific integrated circuit (ASIC), or the like, that is suitable for receiving data, computing, storing, and driving output data and/or signals. The controller 122 may be a device suitable for an embedded application. For example, the controller 122 may include a system on a chip (SOC).

The processor 132 may include one or more processing units. The processor 132 may be a processor of any suitable depth to perform the digital processing requirements disclosed herein. For example, the processor 132 may include a 4-bit processor, a 16-bit processor, a 32-bit processor, a 64-bit processor, or the like.

The memory 134 may include any component or collection of components suitable for storing data. For example, the memory 134 may include volatile memory and/or nonvolatile memory. The memory 134 may include random-access memory (RAM), read-only memory (ROM), erasable programmable read-only memory (EPROM), (electrically erasable programmable read-only memory) EEPROM, flash memory, or the like.

The input/output devices 136 may include any devices suitable for receiving and/or sending information. This information may be in the form of digitally encoded data (from other digital components, for example) and/or analog data (from analog sensors, for example). The input/output devices 136 may include devices such as serial input/output ports, parallel input/output ports, universal asynchronous receiver transmitters (UARTs), discrete logic input/output pins, analog-to-digital converters, digital-to-analog converters. The input/output devices 136 may include specific interfaces with computing peripherals and support circuitry, such as timers, event counters, pulse width modulation (PWM) generators, watchdog circuits, clock generators, and the like. The input/output devices 136 may provide communication within and among the components 120, for example, communication between the controller 122 and the sensors 126, between the controller 122 and the drivers 128, between the controller 122 and the communications interfaces 124, and between the controller and the power management subsystem 130, and as a conduit for any other combination of components 120. The components 120 may support direct communication as well, for example, between a sensor 126 and the power management subsystem 130.

The communications interfaces 124 may include a transmitter 138 and/or a receiver 140. Communication interfaces 124 may include one or more transmitters 138 and/or receivers 140. The transmitter 138 and receiver 140 may include any electrical components suitable for communication to and/or from the electrical components 120. For example, the transmitter 138 and receiver 140 may provide wireline communication and/or wireless communication to devices external to the components 120 and/or external to the device within which the components 120 are integrated.

The transmitter 138 and receiver 140 may enable wireline communication using any suitable communications protocol, for example, protocols suitable for embedded applications. For example, the transmitter 138 and receiver 140 may be configured to enable universal serial bus (USB) communication, Ethernet local-area networking (LAN) communications, and the like.

The transmitter 138 and receiver 140 may enable wireless communications using any suitable communications protocol, for example, protocols suitable for embedded applications. For example, the transmitter 138 and receiver 140 may be configured to enable a wireless personal area network (PAN) communications protocol, a wireless LAN communications protocol, a wide area network (WAN) communications protocol and the like. The transmitter 138 and receiver 140 may be configured to communicate via Bluetooth, for example, with any supported or custom Bluetooth version and/or with any supported or custom protocol, including for example, A/V Control Transport Protocol (AVCTP), A/V Distribution Transport (AVCTP), Bluetooth Network Encapsulation Protocol (BNEP), IrDA Interoperability (IrDA), Multi-Channel Adaptation Protocol (MCAP), and RF Communications Protocol (RFCOMM), and the like. In examples, the transmitter 138 and receiver 140 may be configured to communicate via Bluetooth Low Energy (LE) and/or a Bluetooth Internet of Things (IoT) protocol. The transmitter 138 and receiver 140 may be configured to communicate via local mesh network protocols such as ZigBee, Z-Wave, Thread, and the like, for example. Such protocols may enable the transmitter 138 and receiver 140 to communicate with nearby devices such as the user's cell phone and/or a user's smartwatch. And communication with a local networked device, such as a mobile phone, may enable further communication with other devices across a wide area network (WAN) to devices remote, on the Internet, on a corporate network, and the like.

The transmitter 138 and receiver 140 may be configured to communicate via LAN protocols such as 802.11 wireless protocols like Wi-Fi, including but not limited to, communications in the 2.4 GHz, 5 GHz, and 60 GHz frequency bands. Such protocols may enable the transmitter 138 and receiver 140 to communicate with local network access point, such as a wireless router in a user's home or office, for example. Communication with a local network access point may enable further communication with other devices present on the local network or across a WAN to devices remote, on the Internet, on a corporate network, and the like.

The transmitter 138 and receiver 140 may be configured to communicate via mobile wireless protocols such as global system for mobile communications (GSM), 4G long-term evolution protocol (LTE), 5G, and 5G new radio (NR), and any variety of mobile Internet of things (IoT) protocols. Such protocols may enable the transmitter 138 and receiver 140 to communicate more readily, for example, when a user is mobile, traveling away from home or office, and without manual configuration.

The sensors 126 may include any device suitable for sensing an aspect of its environment such as physical, chemical, mechanical, electrical, encoded information, and the like. The controller 122 may interact with one or more sensors 126. The sensors 126 may include, for example, an oxygen sensor 142, a dose-detection sensor 144, an information sensor 146, a motion sensor 148, and the like. Although not shown, the sensors 126 may include one or more biometric sensors such as a heart rate sensor, a blood oxygen sensor, a blood pressure sensor, a combination thereof, and/or the like.

The oxygen sensor 142 may include any sensing device suitable for determining a presence and/or concentration of oxygen. The oxygen sensor may be a biomimetic-type oxygen sensor, an electrochemical-type oxygen sensor, a semiconductor-type oxygen sensor, or the like. The oxygen sensor 142 may communicate information about the presence and/or concentration of oxygen to the controller 122 via the input/output devices 136.

The dose-detection sensor 144 may be any sensor suitable for detecting a dose of medication that was dispensed. In examples, a mechanical arrangement may translate the force and/or movement that causes dispensing to the sensor 144. The sensor 144 may include a magnetic field sensor, such as a small-scale micro-electromechanical system (MEMS) magnetic field sensor, a contact closure, a reed switch, a potentiometer, a force sensor, a push button, or the like. In examples, the dispensing device may use an electrically controlled dispensing mechanism, like a controllable electric pump. The dose-detection sensor 144 may include a logical determination that the dose was dispensed. The dose-detection sensor 144 may communicate any information suitable for determining dispensing of a dose. For example, the dose-detection sensor 144 may signal a voltage level indicative of a dose, a logic toggle, a numeric dose count, or an analog signal that may be processed (though a lowpass filter, for example) to determine that the signal indicates that a dose delivered to the controller via the input/output devices 136. The dose-detection sensor 144 may have a level of precision or resolution such that the controller 122 may determine the duration of the actuation. For example, an analog signal may be processed via an analog-to-digital converter, processed with a hysteresis threshold, and the resulting state duration may be determined. The dose-detection sensor 144 may be used to measure a dose of medication dispensed by an inhaler, an insulin pump, and/or the like.

The information sensor 146 may include any sensor suitable for reading stored information. In an embedded application with a physical platform, information may be encoded and stored on a variety a media that may be incorporated into aspects of physical design. For example, information about the authenticity, concentration, volume, etc. of a medication that may be dispensed and/or may be associated with the device. In examples, the information may be encoded on a medication container using a quick read (QR) code, in a readable integrated circuit, such as a one-wire identification chip, in a near-field communications (NFC) tag, in physical/mechanical keying, in a Subscriber Identification Module (SIM), or the like. The user may use the device to scan a QR code, and the device may communicate the information to the controller 122 via communications interface 124. In examples, the information sensor 146 may also be suitable for writing information back onto a medium associated with the readable code, such as with a read/writable NFC tag, for example.

The motion sensor 148 may include any sensor suitable for determining relative motion, acceleration, velocity, orientation, and/or the like of the device. The motion sensor 148 may include a piezoelectric, piezoresistive, and/or capacitive component to convert physical motion into an electrical signal. For example, the motion sensor 148 may include an accelerometer. The motion sensor 148 may include a microelectromechanical system (MEMS) device, such as a MEMS thermal accelerometer. The motion sensor 148 may be suitable for sensing a repetitive or periodic motion such as fidgeting by a user holding or wearing the device. The motion sensor 148 may communicate this information via the input/output devices 136 to the processor 132 for processing.

The device may include one or more drivers 128 to communicate feedback to a user and/or to drive a mechanical action. The drivers 128 may include a light emitting diode (LED) driver 152, stepper driver 154, and the like. Other drivers 128 may include haptic feedback drivers, audio output drivers, heating element drivers, and/or the like.

The LED driver 152 may include any circuitry suitable for illuminating an LED. The LED driver 152 may be controllable by the processor 132 via the input/output devices 136. The LED driver 152 may be used to indicate status information to a user. The LED driver 152 may include a multicolor LED driver.

The stepper driver 154 may include any circuitry suitable for controlling a stepper motor. The stepper driver 154 may be controllable by the processor 132 via the input/output driver 136. The stepper driver 154 may be used to control a stepper motor associated with a medical device. In an example, the stepper driver 154 may be used to control a stepper motor of an insulin pump. In an example, the stepper driver 154 may be used to control a motor of a prosthetic arm.

The power management subsystem 130 may include circuitry suitable for managing and distributing power to the components of smart device 120. The power management subsystem 130 may include a battery, a battery charger, and a direct current (DC) power distribution system, for example. The power management subsystem 130 may communicate with the processor 132 via the input/output devices 136 to provide information such as battery charging status. The power management subsystem 130 may include a replaceable battery and/or a physical connector to enable external charging of the battery.

FIG. 2A depicts an example architecture diagram for an example system to support a device, such as a smart device. The system 200 may include the testing device 223, a smartphone 204 with a corresponding app, a smartwatch 206 with corresponding app, a wireless access network 208, a communications network 210, and a computing resource 212.

The smartphone 204 may include an app for providing personalize medical data. The smartphone 204 may provide passive or active tracking and/or location services. The smartphone 204 may collect data regarding the user, process data regarding the user, and/or share data regarding the user. For example, the smartphone 204 may be able to use one of its sensors to collect a biomarker and may be able to share the biomarker data with smartwatch 206, testing device 223, and/or computing resource 212.

The smartwatch 206 may provide a dashboard user interface. The smartwatch 206 may also provide biometric feedback and data such as heart rate and/or heart rate variability, for example. The smartwatch 206 may perform activity tracking and provide activity information. In examples, the smartwatch 206 may include a galvanic skin response sensor.

The testing device 223 may be used for testing, monitoring, and/or determining one or more biomarkers. In an example, testing device 223 may include a sensor for monitoring a biomarker, such as a Philips Biosensor BX100 and the like. In an example, testing device 223 may be a wearable device that may be used for monitoring a heart rate (HR) and/or a heart rate variability (HRV). In an example, testing device 223 may be a compression sock that includes a sensor for determining heat and pressure to monitor a person at risk for diabetes. In an example, testing device 223 may be a device that may be able to dispense a dose of medication, such as an inhaler, an insulin pump, and/or the like. Testing device 223, may be a wearable fitness tracker. Testing device 223 may be an electronic cardiogram (EKG) monitoring device. Testing device 223 may be a blood pressure monitoring device.

The computing resources 212 may provide data storage and processing functionality. The computing resources 212 may receive and analyze behavioral data. For example, the computing resources 212 may receive and analyze behavioral data to identify predictive endpoints for the personalized medical data such as heart rate, heart rate variability, and/or oxygen levels, for example.

The components of the system 200 may communicate with each other over various communications protocols. The device 223 may communicate with a smartphone 204 via a link, such as Bluetooth wireless link 219, for example. The device 223 may communicate with the smartwatch 206 via a link, such as Bluetooth wireless link 221, for example. The smartwatch 206 may communicate with the smartphone 204 over a link, such as a Bluetooth wireless link 216. The smart phone 204 may communicate with the wireless access network 208 over a link, such as wireless link 218. The smartwatch 206 may communicate with the wireless access network 208 over a link, such as wireless link 220. The wireless link 218 and/or wireless link 220 may include any suitable wireless protocol, such as 802.11 wireless protocols like Wi-Fi, GSM, 4G LTE, 5G, and 5G NR, and any variety of mobile IoT protocols.

The communications network 210 may include a long-distance data network, such as a private corporate network, a virtual private network (VPN), a public commercial network, an interconnection of networks, such as the Internet, or the like. The communications network 210 may provide connectivity to the computing resource 212.

The computing resource 212 may include any server resources suitable for remote processing and/or storing of information. For example, the computing resource 212 may include a server, a cloud server, data center, a virtual machine server, and the like. In examples, the device 223 may communicate with the computing resource 212 via the smartphone 204. In examples, the smartwatch 206 may communicate with the computing resource 212 via its own wireless link 220, the smartwatch 206 may communicate with the computing resource 212 via its own wireless link 218, and the device 223 may communicate with the computing resource 212 via its own wireless link 217.

The system 200 may enable the collection and processing of information related to a smoking cessation journey. The system 200 may enable the generation of behavioral support data for presenting personalized medical data, statuses, and/or reporting. For example, an oxygen measurement sensor integrated in the smartwatch 206 may enable convenient oxygen measurements taken during a day. The measurements may be sent and processed by the behavioral support app on the smartphone 204 and/or by the computing resource 212. Analysis of this data may enable identification of a user's mental state, which may be further facilitated by asking the user one or more questions. In an example, a sensor and/or wearable may be used to assess stress or anxiety by proxy, using BP, HR, breathing rate, and the like. In an example, the smartwatch 206, device 223, and/or smartphone 204 may be used with a device 223 to treat stress. In an example, smartwatch 206, device 223, and/or smartphone 204 may be used to track social media to assess depression, bipolar disorder, and the like.

In examples, activity data from the smartwatch 206, from a motion sensor in the device 223, and/or activity tracking by the smartphone 204 can be used to set dynamic thresholds for oxygen levels. The activity data may be used to interpret the oxygen levels more accurately for specific measurements, such as aerobic activity.

FIG. 2B is an example messaging flow diagram for the example system 200. For example, the system 200 may include communication and processing for functions such as initialization and authentication of the testing device and personalized medical data app; data collection from a smartwatch and/or one or more sensors associated with the testing device 223; cloud-based control, triggering, notification messaging and the like, app-based control, messaging and notifications, and the like.

Initialization and authentication messaging 222 may be exchanged between device 202 and the smartphone 204. Initialization and authentication messaging 224 may be exchanged between the computing resource 212 and the smart phone 204. For example, a new user may create a user account via the smartphone 204. The account information may be processed by the computing resource 212. The new user may initialize testing device 223 and/or take steps to authenticate the testing device 223. The information may be communicated via messaging 202 to the smartphone 204 and then via initialization and authentication messaging 224 to computing resources 212. The information may be communicated via initialization and authentication messaging 222 to computing resources 212. Responsive information about user accounts, authentication, and the like may be messaged back to the smartwatch 206 and/or testing device 223.

Data collection functionality may include messaging 226 from the smartwatch 206, and/or testing device 223 to the smartphone 204. This messaging may include information such as activity information, heart rate, heart rate variability, and other biometric information. The data collection functionality may include messaging 228 from the smartwatch 206 and/or testing device 223 to the smartphone 204. The messaging 228 may include information about device operation, such as actuation time/date/location, actuation duration, motion, oxygen level, and the like. In examples, the smartphone 204 may aggregate the messaging 226, 228, process it locally, and/or communicate it or related information to the computing resources 212 via messaging 230.

The system 200 enables cloud-based control functions, app-based control functions, and local control functions. For example, personalized medical data, statuses, and/or reporting may be provided from the computing resources 212 to the smartphone 204 via messaging 232, and if appropriate, from the smartphone 204 to the smartwatch 206 and/or testing device 223 via messaging 234. The computing resource 212 may communicate directly to the smartwatch 206 and/or testing device 223 by using messaging 235.

In examples, personalized medical data, statuses, and/or reporting may be generated from an application and may be displayed at smartphone 204, at smartwatch 206, and/or testing device 223. The application may be on computing resources 212, smartphone 204, smartwatch 206, and/or testing device 223. The personalized medical data, statuses, and/or reporting may be communicated to smartwatch 206 and/or testing device 223 via messaging 236.

In examples, the testing device 223 and/or smartwatch 206 may provide local control via its local processor. Internal system calls and/or local messaging is illustrated as a local loop 238. For example, testing device 223 and/or smartwatch 206 may provide personalized medical data, statuses, and/or reporting.

One or more biomarkers may be provided and/or used by the embodiments described herein. For example, the embodiments described herein may use one or more sensing systems to determine the one or more biomarkers.

A sleep sensing system may measure sleep data, including heart rate, respiration rate, body temperature, movement, and/or brain signals. The sleep sensing system may measure sleep data using a photoplethysmogram (PPG), electrocardiogram (ECG), microphone, thermometer, accelerometer, electroencephalogram (EEG), and/or the like. The sleep sensing system may include a wearable device such as a wristband.

Based on the measured sleep data, the sleep sensing system may detect sleep biomarkers, including but not limited to, deep sleep quantifier, REM sleep quantifier, disrupted sleep quantifier, and/or sleep duration. The sleep sensing system may transmit the measured sleep data to a processing unit. The sleep sensing system and/or the processing unit may detect deep sleep when the sensing system senses sleep data, including reduced heart rate, reduced respiration rate, reduced body temperature, and/or reduced movement. The sleep sensing system may generate a sleep quality score based on the detected sleep physiology.

In an example, the sleep sensing system may send the sleep quality score to a computing system, such as a smart device. In an example, the sleep sensing system may send the detected sleep biomarkers to a computing system, such as a smart device. In an example, the sleep sensing system may send the measured sleep data to a computing system, such as a smart device. The computing system may derive sleep physiology based on the received measured data and generate one or more sleep biomarkers such as deep sleep quantifiers. The computing system may generate a treatment plan, including a pain management strategy, based on the sleep biomarkers. The smart device may detect potential risk factors or conditions, including systemic inflammation and/or reduced immune function, based on the sleep biomarkers.

A core body temperature sensing system may measure body temperature data including temperature, emitted frequency spectra, and/or the like. The core body temperature sensing system may measure body temperature data using some combination of thermometers and/or radio telemetry. The core body temperature sensing system may include an ingestible thermometer that measures the temperature of the digestive tract. The ingestible thermometer may wirelessly transmit measured temperature data. The core body temperature sensing system may include a wearable antenna that measures body emission spectra. The core body temperature sensing system may include a wearable patch that measures body temperature data.

The core body temperature sensing system may calculate body temperature using the body temperature data. The core body temperature sensing system may transmit the calculated body temperature to a monitoring device. The monitoring device may track the core body temperature data over time and display it to a user.

The core body temperature sensing system may process the core body temperature data locally or send the data to a processing unit and/or a computing system. Based on the measured temperature data, the core body temperature sensing system may detect body temperature-related biomarkers, complications and/or contextual information that may include abnormal temperature, characteristic fluctuations, infection, menstrual cycle, climate, physical activity, and/or sleep.

For example, the core body temperature sensing system may detect abnormal temperature based on temperature being outside the range of 36.5° C. and 37.5° C. For example, the core body temperature sensing system may detect post-operation infection or sepsis based on certain temperature fluctuations and/or when core body temperature reaches abnormal levels. For example, the core body temperature sensing system may detect physical activities using measured fluctuations in core body temperature.

For example, the body temperature sensing system may detect core body temperature data and trigger the sensing system to emit a cooling or heating element to raise or lower the body temperature in line with the measured ambient temperature.

In an example, the body temperature sensing system may send the body temperature-related biomarkers to a computing system, such as a smart device. In an example, the body temperature sensing system may send the measured body temperature data to the computing system. The computing system may derive the body temperature-related biomarkers based on the received body temperature data.

A maximal oxygen consumption (VO2 max) sensing system may measure VO2 max data, including oxygen uptake, heart rate, and/or movement speed. The VO2 max sensing system may measure VO2 max data during physical activities, including running and/or walking. The VO2 max sensing system may include a wearable device. The VO2 max sensing system may process the VO2 max data locally or transmit the data to a processing unit and/or a computing system.

Based on the measured VO2 max data, the sensing system and/or the computing system may derive, detect, and/or calculate biomarkers, including a VO2 max quantifier, VO2 max score, physical activity, and/or physical activity intensity. The VO2 max sensing system may select correct VO2 max data measurements during correct time segments to calculate accurate VO2 max information. Based on the VO2 max information, the sensing system may detect dominating cardio, vascular, and/or respiratory limiting factors. Based on the VO2 max information, risks may be predicted including adverse cardiovascular events and/or increased risk of in-hospital morbidity. For example, increased risk of in-hospital morbidity may be detected when the calculated VO2 max quantifier falls below a specific threshold, such as 18.2 ml kg⁻¹ min⁻¹.

In an example, the VO2 max sensing system may send the VO2 max-related biomarkers to a computing system, such as a smart device. In an example, the VO2 max sensing system may send the measured VO2 max data to the computing system. The computer system may derive the VO2 max-related biomarkers based on the received VO2 max data.

A physical activity sensing system may measure physical activity data, including heart rate, motion, location, posture, range-of-motion, movement speed, and/or cadence. The physical activity sensing system may measure physical activity data including accelerometer, magnetometer, gyroscope, global positioning system (GPS), PPG, and/or ECG. The physical activity sensing system may include a wearable device. The physical activity wearable device may include, but is not limited to, a watch, wrist band, vest, glove, belt, headband, shoe, and/or garment. The physical activity sensing system may locally process the physical activity data or transmit the data to a processing unit and/or a computing system.

Based on the measured physical activity data, the physical activity sensing system may detect physical activity-related biomarkers, including but not limited to exercise activity, physical activity intensity, physical activity frequency, and/or physical activity duration. The physical activity sensing system may generate physical activity summaries based on physical activity information.

For example, the physical activity sensing system may send physical activity information to a computing system. For example, the physical activity sensing system may send measured data to a computing system. The computing system may, based on the physical activity information, generate activity summaries, training plans, and/or recovery plans. The computing system may store the physical activity information in user profiles. The computing system may display the physical activity information graphically. The computing system may select certain physical activity information and display the information together or separately.

A respiration sensing system may measure respiration rate data, including inhalation, exhalation, chest cavity movement, and/or airflow. The respiration sensing system may measure respiration rate data mechanically and/or acoustically. The respiration sensing system may measure respiration rate data using a ventilator. The respiration sensing system may measure respiration data mechanically by detecting chest cavity movement. Two or more applied electrodes on a chest may measure the changing distance between the electrodes to detect chest cavity expansion and contraction during a breath. The respiration sensing system may include a wearable skin patch. The respiration sensing system may measure respiration data acoustically using a microphone to record airflow sounds. The respiration sensing system may locally process the respiration data or transmit the data to a processing unit and/or computing system.

Based on measured respiration data, the respiration sensing system may generate respiration-related biomarkers including breath frequency, breath pattern, and/or breath depth. Based on the respiratory rate data, the respiration sensing system may generate a respiration quality score.

Based on the respiration rate data, the respiration sensing system may detect respiration-related biomarkers including irregular breathing, pain, air leak, collapsed lung, lung tissue and strength, and/or shock. For example, the respiration sensing system may detect irregularities based on changes in breath frequency, breath pattern, and/or breath depth. For example, the respiration sensing system may detect pain based on short, sharp breaths. For example, the respiration sensing system may detect an air leak based on a volume difference between inspiration and expiration. For example, the respiration sensing system may detect a collapsed lung based on increased breath frequency combined with a constant volume inhalation. For example, the respiration sensing system may detect lung tissue strength and shock including systemic inflammatory response syndrome (SIRS) based on an increase in respiratory rate, including more than 2 standard deviations. In an example, the detection described herein may be performed by a computing system based on measured data and/or related biomarkers generated by the respiration sensing system.

A blood pressure sensing system may measure blood pressure data including blood vessel diameter, tissue volume, and/or pulse transit time. The blood pressure sensing system may measure blood pressure data using oscillometric measurements, ultrasound patches, photoplethysmography (PPG), and/or arterial tonometry. The blood pressure sensing system using photoplethysmography may include a photodetector to sense light scattered by imposed light from an optical emitter. The blood pressure sensing system using arterial tonometry may use arterial wall applanation. The blood pressure sensing system may include an inflatable cuff, wristband, watch and/or ultrasound patch.

Based on the measured blood pressure data, a blood pressure sensing system may quantify blood pressure-related biomarkers including systolic blood pressure, diastolic blood pressure, and/or pulse transit time. The blood pressure sensing system may use the blood pressure-related biomarkers to detect blood pressure-related conditions such as abnormal blood pressure. The blood pressure sensing system may detect abnormal blood pressure when the measured systolic and diastolic blood pressures fall outside the range of 90/60 to 120-90 (systolic/diastolic). For example, the blood pressure sensing system may detect post-operation septic or hypovolemic shock based on measured low blood pressure. For example, the blood pressure sensing system may detect a risk of edema based on detected high blood pressure. The blood pressure sensing system may predict the required seal strength of a harmonic seal based on measured blood pressure data. Higher blood pressure may require a stronger seal to overcome bursting. The blood pressure sensing system may display blood pressure information locally or transmit the data to a system. The sensing system may display blood pressure information graphically over a period of time.

A blood pressure sensing system may process the blood pressure data locally or transmit the data to a processing unit and/or a computing system. In an example, the detection, prediction and/or determination described herein may be performed by a computing system based on measured data and/or related biomarkers generated by the blood pressure sensing system.

A heart rate variability (HRV) sensing system may measure HRV data including heartbeats and/or duration between consecutive heartbeats. The HRV sensing system may measure HRV data electrically or optically. The HRV sensing system may measure heart rate variability data electrically using ECG traces. The HRV sensing system may use ECG traces to measure the time period variation between R peaks in a QRS complex. An HRV sensing system may measure heart rate variability optically using PPG traces. The HRV sensing system may use PPG traces to measure the time period variation of inter-beat intervals. The HRV sensing system may measure HRV data over a set time interval. The HRV sensing system may include a wearable device, including a ring, watch, wristband, and/or patch.

Based on the HRV data, an HRV sensing system may detect HRV-related biomarkers, complications, and/or contextual information including cardiovascular health, changes in HRV, menstrual cycle, meal monitoring, anxiety levels, and/or physical activity. For example, an HRV sensing system may detect high cardiovascular health based on high HRV. For example, an HRV sensing system may predict stress.

The HRV sensing system may locally process HRV data or transmit the data to a processing unit and/or a computing system. In an example, the detection, prediction, and/or determination described herein may be performed by a computing system based on measured data and/or related biomarkers generated by the HRV sensing system.

The hydration state sensing system may locally process hydration data or transmit the data to a processing unit and/or computing system. In an example, the detection, prediction, and/or determination described herein may be performed by a computing system based on measured data and/or related biomarkers generated by the hydration state sensing system.

A heart rate sensing system may measure heart rate data including heart chamber expansion, heart chamber contraction, and/or reflected light. The heart rate sensing system may use ECG and/or PPG to measure heart rate data. For example, the heart rate sensing system using ECG may include a radio transmitter, receiver, and one or more electrodes. The radio transmitter and receiver may record voltages across electrodes positioned on the skin resulting from expansion and contraction of heart chambers. The heart rate sensing system may calculate heart rate using measured voltage. For example, the heart rate sensing system using PPG may impose green light on skin and record the reflected light in a photodetector. The heart rate sensing system may calculate heart rate using the measured light absorbed by the blood over a period of time. The heart rate sensing system may include a watch, a wearable elastic band, a skin patch, a bracelet, garments, a wrist strap, an earphone, and/or a headband. For example, the heart rate sensing system may include a wearable chest patch. The wearable chest patch may measure heart rate data and other vital signs or critical data including respiratory rate, skin temperature, body posture, fall detection, single-lead ECG, R-R intervals, and step counts. The wearable chest patch may locally process heart rate data or transmit the data to a processing unit. The processing unit may include a display.

Based on the measured heart rate data, the heart rate sensing system may calculate heart rate-related biomarkers including heart rate, heart rate variability, and/or average heart rate. Based on the heart rate data, the heart rate sensing system may detect biomarkers, complications, and/or contextual information including stress, pain, infection, and/or sepsis. The heart rate sensing system may detect heart rate conditions when heart rate exceeds a normal threshold. A normal threshold for heartrate may include the range of 60 to 100 heartbeats per minute. The heart rate sensing system may diagnose post-operation infection, sepsis, or hypovolemic shock based on increased heart rate, including heart rate in excess of 90 beats per minute.

The heart rate sensing system may process heart rate data locally or transmit the data to a processing unit and/or computing system. In an example, the detection, prediction, and/or determination described herein may be performed by a computing system based on measured data and/or related biomarkers generated by the heart rate sensing system. A heart rate sensing system may transmit the heart rate information to a computing system, such as a smart device. The computing system may collect and display cardiovascular parameter information including heart rate, respiration, temperature, blood pressure, arrhythmia, and/or atrial fibrillation. Based on the cardiovascular parameter information, the computing system may generate a cardiovascular health score.

A skin conductance sensing system may measure skin conductance data including electrical conductivity. The skin conductance sensing system may include one or more electrodes. The skin conductance sensing system may measure electrical conductivity by applying a voltage across the electrodes. The electrodes may include silver or silver chloride. The skin conductance sensing system may be placed on one or more fingers. For example, the skin conductance sensing system may include a wearable device. The wearable device may include one or more sensors. The wearable device may attach to one or more fingers. Skin conductance data may vary based on sweat levels.

The skin conductance sensing system may locally process skin conductance data or transmit the data to a computing system. Based on the skin conductance data, a skin conductance sensing system may calculate skin conductance-related biomarkers including sympathetic activity levels. For example, a skin conductance sensing system may detect high sympathetic activity levels based on high skin conductance.

A peripheral temperature sensing system may measure peripheral temperature data including extremity temperature. The peripheral temperature sensing system may include a thermistor, thermoelectric effect, or infrared thermometer to measure peripheral temperature data. For example, the peripheral temperature sensing system using a thermistor may measure the resistance of the thermistor. The resistance may vary as a function of temperature. For example, the peripheral temperature sensing system using the thermoelectric effect may measure an output voltage. The output voltage may increase as a function of temperature. For example, the peripheral temperature sensing system using an infrared thermometer may measure the intensity of radiation emitted from a body's blackbody radiation. The intensity of radiation may increase as a function of temperature.

Based on peripheral temperature data, the peripheral temperature sensing system may determine peripheral temperature-related biomarkers including basal body temperature, extremity skin temperature, and/or patterns in peripheral temperature. Based on the peripheral temperature data, the peripheral temperature sensing system may detect conditions including diabetes.

The peripheral temperature sensing system may locally process peripheral temperature data and/or biomarkers or transmit the data to a processing unit. For example, the peripheral temperature sensing system may send peripheral temperature data and/or biomarkers to a computing system, such as a smart device. The computing system may analyze the peripheral temperature information with other biomarkers, including core body temperature, sleep, and menstrual cycle. For example, the detection, prediction, and/or determination described herein may be performed by a computing system based on measured data and/or related biomarkers generated by the peripheral temperature sensing system.

A respiratory tract bacteria sensing system may measure bacteria data including foreign DNA or bacteria. The respiratory tract bacteria sensing system may use a radio frequency identification (RFID) tag and/or electronic nose (e-nose). The sensing system using an RFID tag may include one or more gold electrodes, graphene sensors, and/or layers of peptides. The RFID tag may bind to bacteria. When bacteria bind to the RFID tag, the graphene sensor may detect a change in signal-to-signal presence of bacteria. The RFID tag may include an implant. The implant may adhere to a tooth. The implant may transmit bacteria data. The sensing system may use a portable e-nose to measure bacteria data.

Based on measured bacteria data, the respiratory tract bacteria sensing system may detect bacteria-related biomarkers including bacteria levels. Based on the bacteria data, the respiratory tract bacteria sensing system may generate an oral health score. Based on the detected bacteria data, the respiratory tract bacteria sensing system may identify bacteria-related biomarkers, complications, and/or contextual information, including pneumonia, lung infection, and/or lung inflammation. The respiratory tract bacteria sensing system may locally process bacteria information or transmit the data to a processing unit. In an example, the detection, prediction, and/or determination described herein may be performed by a computing system based on measured data and/or related biomarkers generated by the respiratory tract bacteria sensing system.

A mental aspect sensing system may measure mental aspect data, including heart rate, heart rate variability, brain activity, skin conductance, oxygenation, skin temperature, galvanic skin response, movement, and/or sweat rate. The mental aspect sensing system may measure mental aspect data over a set duration to detect changes in mental aspect data. The mental aspect sensing system may include a wearable device. The wearable device may include a wristband.

Based on the mental aspect data, the sensing system may detect mental aspect-related biomarkers, including emotional patterns, positivity levels, and/or optimism levels. Based on the detected mental aspect information, the mental aspect sensing system may identify mental aspect-related biomarkers, complications, and/or contextual information including cognitive impairment, stress, anxiety, and/or pain. Based on the mental aspect information, the mental aspect sensing system may generate mental aspect scores, including a positivity score, optimism score, confusion or delirium score, mental acuity score, stress score, anxiety score, depression score, and/or pain score.

Mental aspect data, related biomarkers, complications, contextual information, and/or mental aspect scores may be used to determine a user's potential for a medical condition, such as depression. For example, post-partum depression may be predicted. For example, based on detected positivity and optimism levels, the mental aspect sensing system may determine mood quality and mental state. Based on mood quality and mental state, the mental aspect sensing system may indicate additional care procedures that would benefit a patient, including psychological assistance. For example, based on detected stress and anxiety, the mental aspect sensing system may indicate conditions including anxiety and/or depression. Mental aspect data may include self-report, mini assessment of focus, concentration and/or recall. The metal aspect data may include mini-mental status exam or brain games, psychometric measures, and/or reaction time to a gamified app. The mental aspect data may include speed and errors analysis that may use a smartphone keyboard. The metal aspect data may include voice recognition software for assessing words, pitch, pace, enunciation, and/or the like.

In an example, the detection, prediction, and/or determination described herein may be performed by a computing system based on measured data and/or related biomarkers generated by the mental aspect sensing system. The mental aspect sensing system may process mental aspect data locally or transmit the data to a processing unit.

An autonomic tone sensing system may measure autonomic tone data including skin conductance, heart rate variability, activity, and/or peripheral body temperature. The autonomic tone sensing system may include one or more electrodes, PPG trace, ECG trace, accelerometer, GPS, and/or thermometer. The autonomic tone sensing system may include a wearable device that may include a wristband and/or finger band.

Based on the autonomic tone data, the autonomic tone sensing system may detect autonomic tone-related biomarkers, complications, and/or contextual information, including sympathetic nervous system activity level and/or parasympathetic nervous system activity level. The autonomic tone may describe the basal balance between the sympathetic and parasympathetic nervous system.

In an example, the detection, prediction, and/or determination described herein may be performed by a computing system based on measured data and/or related biomarkers generated by the autonomic tone sensing system. The autonomic tone sensing system may process the autonomic tone data locally or transmit the data to a processing unit.

A circadian rhythm sensing system may measure circadian rhythm data including light exposure, heart rate, core body temperature, cortisol levels, activity, and/or sleep. Based on the circadian rhythm data the circadian rhythm sensing system may detect circadian rhythm-related biomarkers, complications, and/or contextual information including sleep cycle, wake cycle, circadian patterns, disruption in circadian rhythm, and/or hormonal activity.

For example, based on the measured circadian rhythm data, the circadian rhythm sensing system may calculate the start and end of the circadian cycle. The circadian rhythm sensing system may indicate the beginning of the circadian day based on measured cortisol. Cortisol levels may peak at the start of a circadian day. The circadian rhythm sensing system may indicate the end of the circadian day based on measured heart rate and/or core body temperature. Heart rate and/or core body temperature may drop at the end of a circadian day. Based on the circadian rhythm-related biomarkers, the sensing system or processing unit may detect conditions including risk of infection and/or pain. For example, disrupted circadian rhythm may indicate pain and discomfort.

In an example, the detection, prediction, and/or determination described herein may be performed by a computing system based on measured data and/or related biomarkers generated by the circadian rhythm sensing system. The circadian rhythm sensing system may process the circadian rhythm data locally or transmit the data to a processing unit.

A menstrual cycle sensing system may measure menstrual cycle data including heart rate, heart rate variability, respiration rate, body temperature, and/or skin perfusion. Based on the menstrual cycle data, the menstrual cycle unit may indicate menstrual cycle-related biomarkers, complications, and/or contextual information, including menstrual cycle phase. For example, the menstrual cycle sensing system may detect the periovulatory phase in the menstrual cycle based on measured heart rate variability. Changes in heart rate variability may indicate the periovulatory phase. For example, the menstrual cycle sensing system may detect the luteal phase in the menstrual cycle based on measured wrist skin temperature and/or skin perfusion. Increased wrist skin temperature may indicate the luteal phase. Changes in skin perfusion may indicate the luteal phase. For example, the menstrual cycle sensing system may detect the ovulatory phase based on measured respiration rate. Low respiration rate may indicate the ovulatory phase.

In an example, the detection, prediction, and/or determination described herein may be performed by a computing system based on measured data and/or related biomarkers generated by the menstrual cycle sensing system. The menstrual cycle sensing system may locally process menstrual cycle data or transmit the data to a processing unit.

An environmental sensing system may measure environmental data including environmental temperature, humidity, mycotoxin spore count, and airborne chemical data. The environmental sensing system may include a digital thermometer, air sampling, and/or chemical sensors. The environmental sensing system may include a wearable device. The environmental sensing system may use a digital thermometer to measure environmental temperature and/or humidity. The digital thermometer may include a metal strip with a determined resistance. The resistance of the metal strip may vary with environmental temperature. The digital thermometer may apply the varied resistance to a calibration curve to determine temperature. The digital thermometer may include a wet bulb and a dry bulb. The wet bulb and dry bulb may determine a difference in temperature, which then may be used to calculate humidity.

The environmental sensing system may use air sampling to measure mycotoxin spore count. The environmental sensing system may include a sampling plate with adhesive media connected to a pump. The pump may draw air over the plate over a set time at a specific flow rate. The set time may last up to 10 minutes. The environmental sensing system may analyze the sample using a microscope to count the number of spores. The environmental sensing system may use different air sampling techniques including high-performance liquid chromatography (HPLC), liquid chromatography-tandem mass spectrometry (LC-MS/MS), and/or immunoassays and nanobodies.

The environmental sensing system may include chemical sensors to measure airborne chemical data. Airborne chemical data may include different identified airborne chemicals, including nicotine and/or formaldehyde. The chemical sensors may include an active layer and a transducer layer. The active layer may allow chemicals to diffuse into a matrix and alter some physical or chemical property. The changing physical property may include refractive index and/or H-bond formation. The transducer layer may convert the physical and/or chemical variation into a measurable signal, including an optical or electrical signal. The environmental sensing system may include a handheld instrument. The handheld instrument may detect and identify complex chemical mixtures that constitute aromas, odors, fragrances, formulations, spills, and/or leaks. The handheld instrument may include an array of nanocomposite sensors. The handheld instrument may detect and identify substances based on chemical profile.

Based on the environmental data, the sensing system may determine environmental information including climate, mycotoxin spore count, mycotoxin identification, airborne chemical identification, airborne chemical levels, and/or inflammatory chemical inhalation. For example, the environmental sensing system may approximate the mycotoxin spore count in the air based on the measured spore count from a collected sample. The sensing system may identify the mycotoxin spores which may include molds, pollens, insect parts, skin cell fragments, fibers, and/or inorganic particulate. For example, the sensing system may detect inflammatory chemical inhalation, including cigarette smoke. The sensing system may detect second-hand or third-hand smoke.

The environmental sensing system may generate an air quality score based on the measured mycotoxins and/or airborne chemicals. For example, the environmental sensing system may notify about hazardous air quality if it detects a poor air quality score. The environmental sensing system may send a notification when the generated air quality score falls below a certain threshold. The threshold may include exposure exceeding 105 spores of mycotoxins per cubic meter. The environmental sensing system may display a readout of the environment condition exposure over time.

The environmental sensing system may locally process environmental data or transmit the data to a processing unit. In an example, the detection, prediction, and/or determination described herein may be performed by a computing system based on measured data generated by the environmental sensing system.

The biomarker sensing systems may include a wearable device. In an example, the biomarker sensing system may include eyeglasses. The eyeglasses may include a nose pad sensor. The eyeglasses may measure biomarkers, including lactate, glucose, and/or the like. In an example, the biomarker sensing system may include a mouthguard. The mouthguard may include a sensor to measure biomarkers including uric acid and/or the like. In an example, the biomarker sensing system may include a contact lens. The contact lens may include a sensor to measure biomarkers including glucose and/or the like. In an example, the biomarker sensing system may include a tooth sensor. The tooth sensor may be graphene-based. The tooth sensor may measure biomarkers including bacteria and/or the like. In an example, the biomarker sensing system may include a patch. The patch may be wearable on the chest skin or arm skin. For example, the patch may include a chem-phys hybrid sensor. The chem-phys hybrid sensor may measure biomarkers including lactate, ECG, and/or the like. For example, the patch may include nanomaterials. The nanomaterials patch may measure biomarkers including glucose and/or the like. For example, the patch may include an iontophoretic biosensor. The iontophoretic biosensor may measure biomarkers including glucose and/or the like. In an example, the biomarker sensing system may include a microfluidic sensor. The microfluidic sensor may measure biomarkers including lactate, glucose, and/or the like. In an example, the biomarker sensing system may include an integrated sensor array. The integrated sensory array may include a wearable wristband. The integrated sensory array may measure biomarkers including lactate, glucose, and/or the like. In an example, the biomarker sensing system may include a wearable diagnostics device. The wearable diagnostic device may measure biomarkers including cortisol, interleukin-6, and/or the like. In an example, the biomarker sensing system may include a self-powered textile-based biosensor. The self-powered textile-based biosensor may include a sock. The self-powered textile-based biosensor may measure biomarkers including lactate and/or the like.

The various biomarkers described herein may be related to various physiologic systems, including behavior and psychology, cardiovascular system, renal system, skin system, nervous system, GI system, respiratory system, endocrine system, immune system, tumor, musculoskeletal system, and/or reproductive system.

Behavior and psychology may include social interactions, diet, sleep, activity, and/or psychological status. Behavior and psychology-related biomarkers, complications, contextual information, and/or conditions may be determined and/or predicted based on analyzed biomarker sensing systems data. A computing system, as described herein, may select one or more biomarkers (e.g., data from biomarker sensing systems) from behavior and psychology-related biomarkers, including sleep, circadian rhythm, physical activity, nutritional intake and/or mental aspects for analysis. Behavior and psychology scores may be generated based on the analyzed biomarkers, complications, contextual information, and/or conditions. Behavior and psychology scores may include scores for social interaction, diet, sleep, activity, heart rate, blood pressure, respiration, galvanic skin response (GSR), and/or psychological status. For examples, the behavior and phycology scores may be used to assess for anxiety, stress, and the like.

For example, based on the selected biomarker sensing systems data, sleep-related biomarkers, complications, and/or contextual information may be determined, including sleep quality, sleep duration, sleep timing, and/or immune function. Based on the selected biomarker sensing systems data, sleep-related conditions may be predicted, including inflammation. Reduced immune function may be predicted based on disrupted sleep. A compromised immune system may be determined based on analyzed circadian rhythm cycle disruptions.

In an example, sleep metrics may be linked with stress metrics, which may be used to indicate a recommendation to practice meditation and/or deep breathing prior to going to bed. In an example, stress metrics may be able to predict poor sleep if an individual exhibiting these metrics were to try going to sleep without first lowering HR, BP, and other stress/anxiety biometrics.

For example, based on the selected biomarker sensing systems data, activity-related biomarkers, complications, and/or contextual information may be determined, including activity duration, activity intensity, activity type, activity pattern, recovery time, mental health, physical recovery, immune function, and/or inflammatory function. Based on the selected biomarker sensing systems data, activity-related conditions may be predicted. In an example, improved physiology may be determined based on analyzed activity intensity. Moderate intensity exercise may indicate shorter hospital stays, better mental health, better physical recovery, improved immune function, and/or improved inflammatory function. Physical activity type may include aerobic activity and/or non-aerobic activity. Aerobic physical activity may be determined based on analyzed physical activity, including running, cycling, and/or weight training. Non-aerobic physical activity may be determined based on analyzed physical activity, including walking and/or stretching.

For example, based on the selected biomarker sensing systems data, psychological status-related biomarkers, complications, and/or contextual information may be determined (e.g., including stress, anxiety, pain, positive emotions, and/or abnormal states). Based on the selected biomarker sensing systems data, psychological status-related conditions may be predicted, including physical symptoms of disease.

The detection, prediction, determination, and/or generation described herein may be performed by a computing system described herein (e.g., such as a smart device, and/or a computing device) based on measured data and/or related biomarkers generated by the biomarker sensing systems.

The cardiovascular system may include the lymphatic system, blood vessels, blood, and/or heart. Cardiovascular system-related biomarkers, complications, contextual information, and/or conditions may be determined and/or predicted based on analyzed biomarker sensing systems data. Systemic circulation conditions may include conditions for the lymphatic system, blood vessels, and/or blood. A computing system may select one or more biomarkers (e.g., data from biomarker sensing systems) from cardiovascular system-related biomarkers, including blood pressure, VO2 max, hydration state, oxygen saturation, blood pH, sweat, core body temperature, peripheral temperature, edema, heart rate, and/or heart rate variability for analysis.

For example, based on the selected biomarker sensing systems data, lymphatic system-related biomarkers, complications, and/or contextual information may be determined, including swelling, lymph composition, and/or collagen deposition. Based on the selected biomarker sensing systems data, lymphatic system-related conditions may be predicted, including fibrosis, inflammation, and/or post-operation infection. Inflammation may be predicted based on determined swelling. Collagen deposition may be determined based on predicted fibrosis. Increased collagen deposition may be predicted based on fibrosis. Harmonic tool parameter adjustments may be generated based on determined collagen deposition increases. Inflammatory conditions may be predicted based on analyzed lymph composition. Different inflammatory conditions may be determined and/or predicted based on changes in lymph peptidome composition. Metastatic cell spread may be predicted based on predicted inflammatory conditions. Harmonic tool parameter adjustments and margin decisions may be generated based on predicted inflammatory conditions.

For example, based on the selected biomarker sensing systems data, blood vessel-related biomarkers, complications, and/or contextual information may be determined, including permeability, vasomotion, pressure, structure, healing ability, harmonic sealing performance, and/or cardiothoracic health fitness. Based on the selected biomarker sensing systems data, blood vessel-related conditions may be predicted, including infection, anastomotic leak, septic shock and/or hypovolemic shock. In an example, increased vascular permeability may be determined based on analyzed edema, bradykinin, histamine, and/or endothelial adhesion molecules. Endothelial adhesion molecules may be measured using cell samples to measure transmembrane proteins. In an example, vasomotion may be determined based on selected biomarker sensing systems data. Vasomotion may include vasodilators and/or vasoconstrictors. In an example, shock may be predicted based on the determined blood pressure-related biomarkers, including vessel information and/or vessel distribution. Individual vessel structure may include arterial stiffness, collagen content, and/or vessel diameter. Cardiothoracic health fitness may be determined based on VO2 max. Higher risk of complications may be determined and/or predicted based on poor VO2 max.

For example, based on the selected biomarker sensing systems data, blood-related biomarkers, complications, and/or contextual information may be determined, including volume, oxygen, pH, waste products, temperature, hormones, proteins, and/or nutrients. Based on the selected biomarker sensing systems data, blood-related complications and/or contextual information may be determined, including cardiothoracic health fitness, lung function, recovery capacity, anaerobic threshold, oxygen intake, carbon dioxide (CO2) production, fitness, tissue oxygenation, colloid osmotic pressure, and/or blood clotting ability. Based on derived blood-related biomarkers, blood-related conditions may be predicted, including acute kidney injury, hypovolemic shock, acidosis, sepsis, lung collapse, hemorrhage, bleeding risk, infection, and/or anastomotic leak.

For example, an acute kidney injury and/or hypovolemic shock may be predicted based on the hydration state. For example, lung function, lung recovery capacity, cardiothoracic health fitness, anaerobic threshold, oxygen uptake, and/or CO2 product may be predicted based on the blood-related biomarkers, including red blood cell count and/or oxygen saturation. For example, cardiovascular complications may be predicted based on the blood-related biomarkers, including red blood cell count and/or oxygen saturation. For example, acidosis may be predicted based on the pH. Based on acidosis, blood-related conditions may be indicated, including sepsis, lung collapse, hemorrhage, and/or increased bleeding risk. For example, based on sweat, blood-related biomarkers may be derived, including tissue oxygenation. Insufficient tissue oxygenation may be predicted based on high lactate concentration. Based on insufficient tissue oxygenation, blood-related conditions may be predicted, including hypovolemic shock, septic shock, and/or left ventricular failure. For example, based on the temperature, blood temperature-related biomarkers may be derived, including menstrual cycle and/or basal temperature. Based on the blood temperature-related biomarkers, blood temperature-related conditions may be predicted, including sepsis and/or infection. For example, based on proteins, including albumin content, colloid osmotic pressure may be determined. Based on the colloid osmotic pressure, blood protein-related conditions may be predicted, including edema risk and/or anastomotic leak. Increased edema risk and/or anastomotic leak may be predicted based on low colloid osmotic pressure. Bleeding risk may be predicted based on blood clotting ability. Blood clotting ability may be determined based on fibrinogen content. Reduced blood clotting ability may be determined based on low fibrinogen content.

For example, based on the selected biomarker sensing systems data, the computing system may derive heart-related biomarkers, complications, and/or contextual information, including heart activity, heart anatomy, recovery rates, cardiothoracic health fitness, and/or risk of complications. Heart activity biomarkers may include electrical activity and/or stroke volume. Recovery rate may be determined based on heart rate biomarkers. Reduced blood supply to the body may be determined and/or predicted based on irregular heart rate. Slower recovery may be determined and/or predicted based on reduced blood supply to the body. Cardiothoracic health fitness may be determined based on analyzed VO2 max values. VO2 max values below a certain threshold may indicate poor cardiothoracic health fitness. VO2 max values below a certain threshold may indicate a higher risk of heart-related complications.

The detection, prediction, determination, and/or generation described herein may be performed by a computing system described herein, such as a smart device, and/or a computing device based on measured data and/or related biomarkers generated by the biomarker sensing systems.

Renal system-related biomarkers, complications, contextual information, and/or conditions may be determined and/or predicted based on analyzed biomarker sensing systems data. A computing system, as described herein, may select one or more biomarkers (e.g., data from a biomarker sensing systems) from renal system-related biomarkers for analysis. Based on the selected biomarker sensing systems data, renal system-related biomarkers, complications, and/or contextual information may be determined including those related to ureter, urethra, bladder, kidney, general urinary tract, and/or ureter fragility. Based on the selected biomarker sensing systems data, renal system-related conditions may be predicted, including acute kidney injury, infection, and/or kidney stones. In an example, ureter fragility may be determined based on urine inflammatory parameters. In an example, acute kidney injury may be predicted based on analyzed Kidney Injury Molecule-1 (KIM-1) in urine.

The skin system may include biomarkers relating to microbiome, skin, nails, hair, sweat, and/or sebum. Skin-related biomarkers may include epidermis biomarkers and/or dermis biomarkers. Sweat-related biomarkers may include activity biomarkers and/or composition biomarkers. Skin system-related biomarkers, complications, contextual information, and/or conditions may be determined and/or predicted based on analyzed biomarker sensing systems data. A computing system, as described herein, may select one or more biomarkers (e.g., data from biomarker sensing systems) from skin-related biomarkers, including skin conductance, skin perfusion pressure, sweat, autonomic tone, and/or pH for analysis.

For example, based on selected biomarker sensing systems data, skin-related biomarkers, complications, and/or contextual information may be determined, including color, lesions, trans-epidermal water loss, sympathetic nervous system activity, elasticity, tissue perfusion, and/or mechanical properties. Stress may be predicted based on determined skin conductance. Skin conductance may act as a proxy for sympathetic nervous system activity. Sympathetic nervous system activity may correlate with stress. Tissue mechanical properties may be determined based on skin perfusion pressure. Skin perfusion pressure may indicate deep tissue perfusion. Deep tissue perfusion may determine tissue mechanical properties.

Based on selected biomarker sensing systems data, skin-related conditions may be predicted.

For example, based on selected biomarker sensing systems data, sweat-related biomarkers, complications, and/or contextual information may be determined, including activity, composition, autonomic tone, stress response, inflammatory response, blood pH, blood vessel health, immune function, circadian rhythm, and/or blood lactate concentration. Based on selected biomarker sensing systems data, sweat-related conditions may be predicted, including ileus, cystic fibrosis, diabetes, metastasis, cardiac issues, and/or infections.

For example, sweat composition-related biomarkers may be determined based on selected biomarker data. Sweat composition biomarkers may include proteins, electrolytes, and/or small molecules. Based on the sweat composition biomarkers, skin system complications, conditions, and/or contextual information may be predicted, including ileus, cystic fibrosis, acidosis, sepsis, lung collapse, hemorrhage, bleeding risk, diabetes, metastasis, and/or infection. For example, based on protein biomarkers, including sweat neuropeptide Y and/or sweat antimicrobials, stress response may be predicted. Higher sweat neuropeptide Y levels may indicate greater stress response. Cystic fibrosis and/or acidosis may be predicted based on electrolyte biomarkers, including chloride ions, pH, and other electrolytes. High lactate concentrations may be determined based on blood pH. Acidosis may be predicted based on high lactate concentrations. Sepsis, lung collapse, hemorrhage, and/or bleeding risk may be predicted based on predicted acidosis. Diabetes, metastasis, and/or infection may be predicted based on small molecule biomarkers. Small molecule biomarkers may include blood sugar and/or hormones. Hormone biomarkers may include adrenaline and/or cortisol. Based on predicted metastasis, blood vessel health may be determined. Infection due to lower immune function may be predicted based on detected cortisol. Lower immune function may be determined and/or predicted based on high cortisol. For example, sweat-related conditions, including stress response, inflammatory response, and/or ileus, may be predicted based on determined autonomic tone. Greater stress response, greater inflammatory response, and/or ileus may be determined and/or predicted based on high sympathetic tone.

The respiratory system may include the upper respiratory tract, lower respiratory tract, respiratory muscles, and/or system contents. The upper respiratory tract may include the pharynx, larynx, mouth and oral cavity, and/or nose. The lower respiratory tract may include the trachea, bronchi, alveoli, and/or lungs. The respiratory muscles may include the diaphragm and/or intercostal muscles. Respiratory system-related biomarkers, complications, contextual information, and/or conditions may be determined and/or predicted based on analyzed biomarker sensing systems data. A computing system, as described herein, may select one or more biomarkers (e.g., data from biomarker sensing systems) from respiratory system-related biomarkers, including bacteria, coughing and sneezing, respiration rate, VO2 max, and/or activity for analysis.

The upper respiratory tract may include the pharynx, larynx, mouth and oral cavity, and/or nose. For example, based on the selected biomarker sensing systems data, upper respiratory tract-related biomarkers, complications, and/or contextual information may be determined. Based on the selected biomarker sensing systems data, upper respiratory tract-related conditions may be predicted, including SSI, inflammation, and/or allergic rhinitis. In an example, SSI may be predicted based on bacteria and/or tissue biomarkers. Bacteria biomarkers may include commensals and/or pathogens. Inflammation may be indicated based on tissue biomarkers. Mucosa inflammation may be predicted based on nose biomarkers, including coughing and sneezing. General inflammation and/or allergic rhinitis may be predicted based on mucosa biomarkers. Mechanical properties of various tissues may be determined based on systemic inflammation.

The lower respiratory tract may include the trachea, bronchi, alveoli, and/or lungs. For example, based on the selected biomarker sensing systems data, lower respiratory tract-related biomarkers, complications, and/or contextual information may be determined, including bronchopulmonary segments. Based on the selected biomarker sensing systems data, lower respiratory tract-related conditions may be predicted.

Based on the selected biomarker sensing systems data, lung-related biomarkers, complications, and/or contextual information may be determined. Lung-related biomarkers may include lung respiratory mechanics, lung disease, lung mechanical properties, and/or lung function. Lung respiratory mechanics may include total lung capacity (TLC), tidal volume (TV), residual volume (RV), expiratory reserve volume (ERV), inspiratory reserve volume (IRV), inspiratory capacity (IC), inspiratory vital capacity (IVC), vital capacity (VC), functional residual capacity (FRC), residual volume expressed as a percent of total lung capacity (RV/TLC %), alveolar gas volume (VA), lung volume (VL), forced vital capacity (FVC), forced expiratory volume over time (FEVt), difference between inspired and expired carbon monoxide (DLco), volume exhaled after first second of forced expiration (FEV1), forced expiratory flow related to portion of functional residual capacity curve (FEFx), maximum instantaneous flow during functional residual capacity (FEFmax), forced inspiratory flow (FIF), highest forced expiratory flow measured by peak flow meter (PEF), and maximal voluntary ventilation (MVV).

TLC may be determined based on lung volume at maximal inflation. TV may be determined based on volume of air moved into or out of the lungs during quiet breathing. RV may be determined based on volume of air remaining in lungs after a maximal exhalation. ERV may be determined based on maximal volume inhaled from the end-inspiratory level. IC may be determined based on aggregated IRV and TV values. IVC may be determined based on maximum volume of air inhaled at the point of maximum expiration. VC may be determined based on the difference between the RV value and TLC value. FRC may be determined based on the lung volume at the end-expiratory position. FVC may be determined based on the VC value during a maximally forced expiratory effort. MVV may be determined based on the volume of air expired in a specified period during repetitive maximal effort.

Based on the selected biomarker sensing systems data, lung-related conditions may be predicted, including emphysema, chronic obstructive pulmonary disease, chronic bronchitis, asthma, cancer, and/or tuberculosis. Lung diseases may be predicted based on analyzed spirometry, x-rays, blood gas, and/or diffusion capacity of the alveolar capillary membrane. Lung diseases may narrow airways and/or create airway resistance. Lung cancer and/or tuberculosis may be detected based on lung-related biomarkers, including persistent coughing, coughing blood, shortness of breath, chest pain, hoarseness, unintentional weight loss, bone pain, and/or headaches. Tuberculosis may be predicted based on lung symptoms including coughing for 3 to 5 weeks, coughing blood, chest pain, pain while breathing or coughing, unintentional weight loss, fatigue, fever, night sweats, chills, and/or loss of appetite.

The detection, prediction, determination, and/or generation described herein may be performed by a computing system described herein, such as a smart device, a computing system, and/or a smart device based on measured data and/or related biomarkers generated by the biomarker sensing systems.

As disclosed herein, health data and/or biometric data may be captured using a number of devices. The health data and/or biometric data may be analyzed and/or processed using artificial intelligence (AI) and/or machine learning (ML). In an example, AI and/or ML may be used to make tailored recommendations to the individual. In an example, AI and/or ML may be used to enhance software by learning and conveying what recommendations may or may not be working for an individual, for typologies, for groups, and/or for normative populations.

Machine learning is a branch of artificial intelligence that seeks to build computer systems that may learn from data without human intervention. These techniques may rely on the creation of analytical models that may be trained to recognize patterns within a dataset, such as a data collection. These models may be deployed to apply these patterns to data, such as biomarkers, to improve performance without further guidance.

Machine learning may be supervised (e.g., supervised learning). A supervised learning algorithm may create a mathematical model based on training a dataset (e.g., training data). The training data may consist of a set of training examples. A training example may include one or more inputs and one or more labeled outputs. The labeled output(s) may serve as supervisory feedback. In a mathematical model, a training example may be represented by an array or vector, sometimes called a feature vector. The training data may be represented by row(s) of feature vectors, constituting a matrix. Through iterative optimization of an objective function (e.g., cost function), a supervised learning algorithm may learn a function (e.g., a prediction function) that may be used to predict the output associated with one or more new inputs. A suitably trained prediction function may determine the output for one or more inputs that may not have been a part of the training data. Example algorithms may include linear regression, logistic regression, and neural network. Example problems solvable by supervised learning algorithms may include classification, regression problems, and the like.

Machine learning may be unsupervised (e.g., unsupervised learning). An unsupervised learning algorithm may train on a dataset that may contain inputs and may find a structure in the data. The structure in the data may be similar to a grouping or clustering of data points. As such, the algorithm may learn from training data that may not have been labeled. Instead of responding to supervisory feedback, an unsupervised learning algorithm may identify commonalities in training data and may react based on the presence or absence of such commonalities in each train example. Example algorithms may include Apriori algorithm, K-Means, K-Nearest Neighbors (KNN), K-Medians, and the like. Example problems solvable by unsupervised learning algorithms may include clustering problems, anomaly/outlier detection problems, and the like.

Machine learning may include reinforcement learning, which may be an area of machine learning concerned with how software agents may take actions in an environment to maximize a notion of cumulative reward. Reinforcement learning algorithms may not assume knowledge of an exact mathematical model of the environment (e.g., represented by Markov decision process (MDP)) and may be used when exact models may not be feasible. Reinforcement learning algorithms may be used in autonomous vehicles or in learning to play a game against a human opponent.

Machine learning may be a part of a technology platform called cognitive computing (CC), which may constitute various disciplines such as computer science and cognitive science. CC systems may be capable of learning at scale, reasoning with purpose, and interacting with humans naturally. By means of self-teaching algorithms that may use data mining, visual recognition, and/or natural language processing, a CC system may be capable of solving problems and optimizing human processes.

The output of machine learning's training process may be a model for predicting outcome(s) on a new dataset. For example, a linear regression learning algorithm may be a cost function that may minimize the prediction errors of a linear prediction function during the training process by adjusting the coefficients and constants of the linear prediction function. When a minimal is reached, the linear prediction function with adjusted coefficients may be deemed trained and constitute the model the training process has produced. For example, a neural network (NN) algorithm (e.g., multilayer perceptrons (MLP)) for classification may include a hypothesis function represented by a network of layers of nodes that are assigned with biases and interconnected with weight connections. The hypothesis function may be a non-linear function (e.g., a highly non-linear function) that may include linear functions and logistic functions nested together with the outermost layer consisting of one or more logistic functions. The NN algorithm may include a cost function to minimize classification errors by adjusting the biases and weights through a process of feedforward propagation and backward propagation. When a global minimum may be reached, the optimized hypothesis function with its layers of adjusted biases and weights may be deemed trained and constitute the model the training process has produced.

Data collection may be performed for machine learning as a first stage of the machine learning lifecycle. Data collection may include steps such as identifying various data sources, collecting data from the data sources, integrating the data, and the like. For example, for training a machine learning model for predicting medical issues and/or complications. Data sources that include medical data, such as a patient's medical conditions and biomarker measurement data, may be identified. Such data sources may be a patient's electronic medical records (EMR), a computing system storing the patient's pre-biomarker measurement data, and/or other like datastores. The data from such data sources may be retrieved and stored in a central location for further processing in the machine learning lifecycle. The data from such data sources may be linked (e.g., logically linked) and may be accessed as if they were centrally stored. Medical data may be similarly identified and/or collected. Further, the collected data may be integrated. In examples, a patient's medical record data, biomarker measurement data, and/or other medical data may be combined into a record for the patient. The record for the patient may be an EMR.

Data preparation may be performed for machine learning as another stage of the machine learning lifecycle. Data preparation may include data preprocessing steps such as data formatting, data cleaning, and data sampling. For example, the collected data may not be in a data format suitable for training a model. In an example, a patient's integrated data record of EMR data and biomarker measurement data may be in a rational database. Such data record may be converted to a flat file format for model training. In an example, the patient's EMR data may include medical data in text format, such as the patient's diagnoses of emphysema, treatment (e.g., chemotherapy, radiation, blood thinner). Such data may be mapped to numeric values for model training. For example, the patient's integrated data record may include personal identifier information or other information that may identifier a patient such as an age, an employer, a body mass index (BMI), demographic information, and the like. Such identifying data may be removed before model training. For example, identifying data may be removed for privacy reasons. As another example, data may be removed because there may be more data available than may be used for model training. In such case, a subset of the available data may be randomly sampled and selected for model training and the remainder may be discarded.

Data preparation may include data transforming procedures (e.g., after preprocessing), such as scaling and aggregation. For example, the preprocessed data may include data values in a mixture of scales. These values may be scaled up or down, for example, to be between 0 and 1 for model training. For example, the preprocessed data may include data values that carry more meaning when aggregated. In an example, there may be multiple prior colorectal procedures a patient has had. The total count of prior colorectal procedures may be more meaningful for training a model to predict complications due to adhesions. In such case, the records of prior colorectal procedures may be aggregated into a total count for model training purposes.

Model training may be another aspect of the machine learning lifecycle. The model training process as described herein may be dependent on the machine learning algorithm used. A model may be deemed suitably trained after it has been trained, cross validated, and tested. Accordingly, the dataset from the data preparation stage (e.g., an input dataset) may be divided into a training dataset (e.g., 60% of the input dataset), a validation dataset (e.g., 20% of the input dataset), and a test dataset (e.g., 20% of the input dataset). After the model has been trained on the training dataset, the model may be run against the validation dataset to reduce overfitting. If accuracy of the model were to decrease when run against the validation dataset when accuracy of the model has been increasing, this may indicate a problem of overfitting. The test dataset may be used to test the accuracy of the final model to determine whether it is ready for deployment or whether more training is required.

Model deployment may be another aspect of the machine learning lifecycle. The model may be deployed as a part of a standalone computer program. The model may be deployed as a part of a larger computing system. A model may be deployed with model performance parameter(s). Such performance parameters may monitor the model accuracy as it is used for predicating on a dataset in production. For example, such parameters may keep track of false positives and false negatives for a classification model. Such parameters may further store the false positives and false negatives for further processing to improve the model's accuracy.

Post-deployment model updates may be another aspect of the machine learning cycle. For example, a deployed model may be updated as false positives and/or false negatives are predicted on production data. In an example, for a deployed multilayer perceptions (MLP) model for classification, as false positives occur, the deployed MLP model may be updated to increase the probably cutoff for predicting a positive to reduce false positives. In an example, for a deployed MLP model for classification, as false negatives occur, the deployed MLP model may be updated to decrease the probability cutoff for predicting a positive to reduce false negatives. In an example, for a deployed MLP model for classification of medical issues and/or complications, as both false positives and false negatives occur, the deployed MLP model may be updated to decrease the probability cutoff for predicting a positive to reduce false negatives because it may be less critical to predict a false positive than a false negative.

For example, a deployed model may be updated as more live production data become available as training data. In such case, the deployed model may be further trained, validated, and tested with such additional live production data. In an example, the updated biases and weights of a further-trained MLP model may update the deployed MLP model's biases and weights. Those skilled in the art recognize that post-deployment model updates may not be a one-time occurrence and may occur as frequently as suitable for improving the deployed model's accuracy.

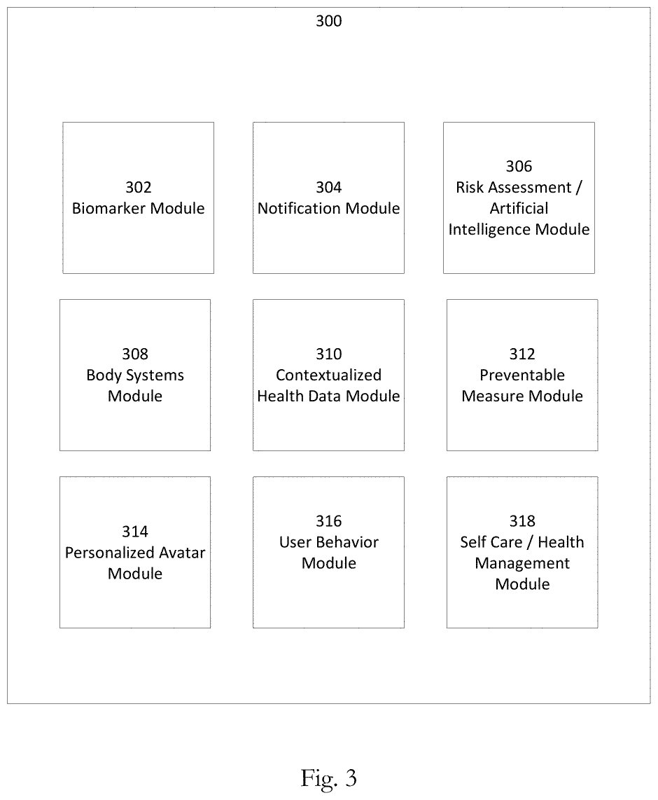

FIG. 3 depicts a block diagram 300 of an example device that may include one or more modules (e.g., software modules) for providing personalized medical data, statuses, and/or recommendations. The block diagram 300 may include a biomarker module 302, a notification module 304, a risk assessment/artificial intelligence module 306, a body systems module 308, a contextualized health data module 310, a preventative measure module 312, a personalized avatar module 314, a user behavior module 316, and/or a self-care/health management module 318.

The biomarker module 302 may detect biomarkers to help identify whether a user may be at risk for one or more diseases. The biomarker module 302 may include biomarkers used with different sensing systems and different physiologic systems. For example, the biomarkers may be any of the biomarkers, sensing systems, and/or physiologic systems may be any of the biomarkers, sensing systems, and/or physiologic systems described herein. The one or more sensing systems may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, and/or the like. The one or more sensors may measure the biomarkers as described herein using one of more of the following sensing technologies: photoplethysmography, electrocardiography, electroencephalography, colorimetry, impedimentary, potentiometry, amperometry, etc. In examples, the sensing systems may include wearable sensing systems.

The one or more sensors may be configured for sensing one or more biomarker parameters associated with specific health issues. The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the biomarker module 302. The information from the biomarkers may be determined and/or used by the biomarker module 302 to improve said systems and/or to improve patient outcomes, for example.

The biomarker parameters may be used to provide biomarker data to individuals, medical professionals, and/or hospitals. The biomarker data may be collected to show current health conditions. The biomarker data may be evaluated in relation to normal levels. A combination of biomarkers may (e.g., may also) be used to evaluate certain health conditions. In examples, one biomarker may not be enough to evaluate health conditions. In some cases, one biomarker that may indicate certain health conditions alone may be used to indicate different health conditions when combined with other biomarkers. The biomarker module 302 may refer the user to a doctor to get an in-depth diagnosis if it detects a problem, comparing the biomarker values received to the expected biomarker values. Over time, the biomarker module 302 may receive more data, which may allow it to become smarter as the data set gets larger. This may allow for better integration of conditions.

In examples, the biomarker module 302 may be located in a cloud, on a server, as an application on a smart device, within a wearable device, a combination thereof, and/or the like. The biomarker module 302 may share data with other devices. The biomarker module 302 may help engage people that are using it and may help them stay interested in the details (e.g., scientific details) that may be provided.

The notification module 304 may provide healthcare information to users. In examples, the healthcare information may be presented via a personal dashboard. The personal dashboard may update on a regular (e.g., daily) basis, for example, with real time notifications on specific health issues that may emerge. The notification module 304 may allow a user to manage their health and/or prevent a health issue (e.g., a more serious health issue) from occurring. The notification module 304 may be able to identify body parts in a gamification mechanism as a way to get users in touch with their health. The notification module 304 may provide the different points of information in an engaging, instructive manner. Rather than having a black and white series of numbers and ranges, the notification module 304 may use color coding and visuals for users, making users more likely to read and engage with their information, to remember their information, find their information valuable, and utilize their information.

The notification module 304 may also serve as an alert system (e.g., via a check engine light). In examples, if the biomarker module 302 detects abnormal data or abnormal sources of data, a body part where the abnormal data is occurring may light up like an icon alert. The icon alert may tell the user to pay attention to the abnormalities now, as well as provide a self-generated exploration about the user's health, the user's body parts, and the user's well-being.

The risk assessment/artificial intelligence module 306 may receive information from the biomarker module 302 and help identify users at risk for certain diseases. In examples, if a user is a smoker, and the user's lung health is a focus, biomarkers of lung cancer risk may be combined with other biomarkers and behavioral indices determined from the biomarker module 302. The risk assessment/artificial intelligence module 306 may take the biomarkers from the biomarker module 302 and provide information to the user regarding lung cancer and/or other health risks. This may, for example, provide users with a more engaging way of taking charge of their health.

In examples, a user may wear a compression sock that people at risk for diabetes would wear. In that compression sock, the biomarker module 302 may gauge heat and pressure. The risk assessment/artificial intelligence module 306 may use the digital interface of the application to pair it with that device to help detect diabetes and blood clots in the leg. The risk assessment/artificial intelligence module 306 may (e.g., may also) relate to blood clots and issues with the lung and the heart, providing a system that may have a framework adapted for specific conditions, general organ challenges, or specific devices and technologies as they emerge.

Over time, the risk assessment/artificial intelligence module 306 may receive more data, allowing it to become smarter as the data set gets larger. This may allow for better integration of conditions. In examples, if detecting lung cancer risk for smokers (e.g., via breath sensors and genetic testing), then heart health, risk of stroke, and hypertension may (e.g., may also) be considered (e.g., along with the lung cancer risk or diagnosis).

The risk assessment/artificial intelligence module 306 may perform types of screening or risk assessment that may be quantitative in nature and/or may be psychometric in nature such that it makes specific recommendations to improve health or manage pain, for example. The risk assessment/artificial intelligence module 306 may function as a personal digital assistant (PDA) or smart device that captures information in real time. For example, if information may be captured during the day before a user goes to sleep, when the user wakes up in the morning, they may observe a sound quality sleep of 6.8 hours overnight, for example. The sound quality sleep may be compared to the day before, week before, etc. For example, if a user is mildly dehydrated, the risk assessment/artificial intelligence module 306 may encourage the user to drink more water and reduce morning caffeine consumption. The risk assessment/artificial intelligence module 306 may refer the user to a doctor to get an in-depth diagnosis if it detects a problem, which may be based on information received from the biomarker module 302.

In examples, the risk assessment/artificial intelligence module 306 may start analyzing data related to a specific health condition. The data may be received from the biomarker module 302. Some data may be related to biomarkers that have been identified and some data may be related to biomarkers that are still to be researched. Users may receive the data and take actionable steps to manage their health condition and/or prevent something from a health risk perspective. In examples, the data related to the specific health condition may be applied to other health conditions. Eventually, the conditions may integrate where appropriate, capturing a larger amount of data over time, in which the risk assessment/artificial intelligence module 306 may become a big data artificial intelligence system. This approach may allow the risk assessment/artificial intelligence module 306 to detect hidden health problems, just from a general health intervention.

In examples, the risk assessment/artificial intelligence module 306 may be used to inspect, adjust, correct, and/or filter data. The risk assessment/artificial intelligence module 306 may be used to scrub data to remove errors in data, to improve the accuracy of the data, and/or the like. The risk assessment/artificial intelligence module 306 may be used to detect errors in data, such as errors in biometric data. The risk assessment/artificial intelligence module 306 may correct the detected errors in the data, may remove the detected errors in the data, may notify a user of the detected errors in the data, and/or the like.

The body systems module 308 may determine a body system and/or an organ context for the user when the user clicks on their personal avatar. A body system may be a system of the human body such the circulatory system, the digestive system, the excretory system, the endocrine system, the integumentary system, the exocrine system, the immune system, the lymphatic system, the muscular system, the nervous system, the renal system, the urinary system, the reproductive system, the respiratory system, the skeletal system, and/or the like. An organ context may indicate a context of one or more organs that may be associated with a body system based on a location, a biomarker, a disease, and/or the like. For example, an organ context associated with chest pain may include the heart and lungs. As another example, an organ context associated with abdominal pain may include the intestines, the stomach, the pancreas, and the like.

The body systems module 308 may determine one or more biomarkers that are related to what the user has clicked on. The body systems module 308 may target a specific body system, a group of body systems, a body system related to an organ, a specific organ, a group of related organs, and/or a group of organs that might be related to biomarker data received from the biomarker module 302. In examples, which organs to link together may be determined by the body systems module 308 based on the biomarker data received from the biomarker data module 302. For example, if a user received biomarker data from the biomarker module 302 that tells them something about blood pressure, it may tell them about pulmonary function, but may also tell them about their heart. As such, the body systems module 308 would link these together. In examples, organs may be linked to each other (e.g., may form an organ context) based on a shared association with a location (e.g., an area of the human body), a biomarker, and/or a disease.

In examples, the body systems module 308 may determine a body system and/or an organ context by displaying a personal avatar to a user. The user may customize the avatar to increase engagement (e.g., the avatar is not anonymous, but personalized to each user). The personalized avatar may display the internal organs and/or body parts of the user. The body systems module 308 may receive a user selection and indicate a body system and/or an organ context. The user may see the avatar and click on a portion of the avatar. The body systems module 308 may display the body system and/or the organ contexts to the user based on the portion clicked on. The user may select the body system and/or the organ context from the one or more organ contexts. The body system and/or the organ context may be associated with a body system, a group of body systems, a specific organ, a group of related organs, and/or a group of organs related to a biomarker (e.g., blood pressure with dizziness may be related to the heart and/or brain). In examples, the body systems module 308 may determine a biomarker related to the body system and/or the organ context based on the biomarker information received from the biomarker module 302. As discussed above, the biomarker data may be determined from the biomarker module 302 using another device, such as a wearable device, a medical device and/or instrument (e.g., EKG, x-ray, glucose monitor, etc.). The biomarker data may come from a database of individual health data and/or population health data.

The contextualized health data module 310 may filter healthcare data to make it relevant to the user based on their selections and understanding of the context they are looking at, using the user selection to make sense of the data itself. The contextualized health data module 310 may generate contextualized health for the organ context that indicates a significance of the biomarker. In examples, the contextualized health data module 310 may determine a significance of the biomarker by comparing the biomarker to a threshold. In examples, the contextualized health data module 310 may compare the biomarker against a model associated with a particular disease. The model may be an artificial intelligence model (e.g., pretrained neural network, etc.) or a risk model (e.g., if the patient has heart disease, the biomarker may indicate that the patient is at risk for a heart attack). The contextualized health data module 310 may display the biomarker data within a range. The range may show what is considered normal and/or healthy for the user. The contextualized health data module 310 may display contextualized health data that shows the biomarker along with other relevant medical data. The contextualized health data module 310 may display the biomarker data with an indication of the likelihood of a negative outcome (e.g., the biomarker data indicates a user 50% more likely to develop heart disease).

The contextualized health data module 310 may detect and/or resolve conflicting data. For example, data associated with one or more biomarkers may conflict with each other and may indicate different results and/or diagnosis. The contextual health data module 310 may detect the conflict between the biomarkers and may resolve the conflict data through analysis. For example, the contextual health data module 310 may analyze the conflicting data (e.g., biomarkers) and may determine that the most likely cause may be due to a bad sensor.

The contextual health data module 310 may analyze the conflict data using historical data. The contextual health data module 310 may retrieve the EMR data for a patient, may compare the conflicting biomarker data to the EMR data, and may resolve the conflicting data based on the EMR data. For example, contextual health data module 310 may determine that the EMR data indicates that a patient has a heart condition and may dismiss and/or ignore a biomarker that indicates that the patient does not have a heart condition.

The preventative measure module 312 may display a preventative measure (e.g., a recommended action) to improve a health issue. The preventative measures may be based off biomarker data, contextual data, organ context, etc. The health issue may be related to the organ context. The preventative measure module 312 may display an action that may assist in moving the biomarker below a threshold. The preventative measure module 312 may display an action that improves overall health. In examples, if a user is at risk for developing type two diabetes but they do not have diabetes yet, there may be metrics that indicate glucose levels, or that indicate to other things (e.g., such as pre-diabetes weight issues) that are associated with pre-diabetes, to help prevent the patient from developing diabetes. The preventative measure module 312 may make a series of recommendations around nutrition, diet, exercise, and/or the like, to help prevent users from developing the condition that they are at risk of developing. In examples, if biomarker data or a psychometric screening identified that a pregnant woman is at an elevated risk for perinatal depression and it is determined that she is having sleep trouble, she is under physical stress, and/or the like, then the system may offer suggestions to prevent the perinatal depression from manifesting.

In examples, the preventative measure module 312 may determine behavioral biomarkers. Some data related to behavior may be compared on a smartphone and may not be captured in a traditional physiological way. For example, users may be prompted to answer a number of questions (e.g., mental health questions) that indicate stress level or quality of sleep the night before, anxiety, and/or the like, which may be recorded on a smartphone or other personal device. The responses to the questions (e.g., a user input including user responses to the mental health questions) may be compared against normative data and the preventative measure module 312 may make predictions about whether users either have a condition or are at risk. The system may prompt the user to answer questions m (e.g., on a daily basis) to detect how a user is feeling that day and may give the user some feedback about the environment.

The personalized avatar module 314 may provide a graphic of a human body that may be personalized into a personal avatar. A user may tap on different body parts of the personal avatar to render the data/information that may be relevant to that body part. In examples, tapping the chest area may visualize the heart, and another tap may show the status of one or more heart measurements such as a current heart rate, a heart rate trend, a comparison to normal/healthy heart rate range, and/or the like. Users may click further to get tips, suggestions, and techniques on health related to a body part. After the body part is tapped, the personalized avatar module 314 may provide biomarker information regarding that body part. In examples, if a user has a stomachache, they may tap on the stomach. The stomach biomarkers may then pop up and tell the user they have been drinking too much alcohol, for example. As such, the personalized avatar module 314 may be more interesting for people that do not know much about biomarkers, since the user is able to see (e.g., via the avatar) a depiction of how the biomarker relates to their body.

The personalized avatar module 314 may provide prediction assessments when looking at demographics and other information, incorporating some biomarker data, and/or the like. Personalized recommendations may be provided for users, such as provided suggestions of actions to take or avoid. The recommendations may entice users and help the user understand how the recommended actions may have provided health benefits. In examples, users may be provided estimates of how many days of life they may add by taking or avoiding an action (e.g., by quitting smoking today, by taking a daily aspirin, and/or the like). The personalized avatar module 314 may integrate one organ system with another. In examples, if a user is a smoker, and the user's lung health was a focus, biomarkers of lung cancer risk may be combined with other biomarkers and behavioral indices, which may provide information to the user regarding lung cancer (and possibly other health risks). Therefore, users may have a more engaging way of taking charge of their health.

The personalized avatar module 314 may explain data back to a user which may be actionable through color coding and simplistic approaches. For example, if a user has a headache and they tap on their brain, but their issue is head pressure, the application may describe blood pressure and the impact on headache. Color may be used to describe and/or indicate a degree of a biometric. Color may be used to describe and/or indicate a severity of an issue. For example, a slightly elevated blood pressure may be represented as purple, an elevated blood pressure may be represented as red, and a normal blood pressure may be represented as blue. Such representations may be useful for demonstrating how a biometric parameter may affect the patient, even when the patient may not be aware. For example, patients are often unable to determine whether they have high blood pressure, but color baby used as an indication of where their blood pressure may be. And another example, a numeric indicator may be used. For example, if instead the user's issue is that they are taking their blood pressure reading, and they are concerned with their blood pressure number, the personalized avatar module 314 may describe managing their hypertension or their diabetes. The personalized avatar module 314 may output different recommendations based on different content (e.g., contexts) that may emerge (e.g., based on whether a user is concerned with a headache or with high blood pressure) even if the data is the same.

The user behavior module 316 may track and analyze user behavior in real time based on the biomarker data received from the biomarker module 302. In examples, the user behavior module 316 may initiate event triggers if biomarker data is outside of an expected range. The event trigger may correspond to values of a biomarkers being over or under threshold values. In examples, the threshold values may differ while the user is performing certain activities. In examples, the values of the biomarkers may be a set of values in a recovery timeline after the user undergoes surgery or a medical procedure. If the actual biomarker data received from the biomarker module 302 includes values over or under the threshold values, the user behavior module 316 may trigger the event trigger. If the event trigger occurs, the notification module 304 may generate a notification alert corresponding to the event trigger. In examples, the notification module 304 may provide notifications to users. In examples, the notification module 304 may provide notifications to different caregivers or hospitals (e.g., if the event trigger is serious). The notification alert may indicate an emergency and that immediate action should be taken. The notification alert may be a unique notification tailored for a specific patient. The user behavior module 316 may monitor certain behaviors that lead to biomarker data being outside of the expected range, which may help mitigate future event triggers.

In examples, the user behavior module 316 may monitor if biomarker datapoints received by the biomarker module 302 fall within a desired range for users. In examples, the desired range may be associated with a recovery threshold for patients after surgery. The desired ranges of biomarkers may change while users are performing different activities, such as exercising. The recovery threshold may be a patient-monitored event that initiates an elevated risk to users. The recovery threshold may cause the notification module 304 to notify users of the recovery event triggers. If the biomarker data received by the biomarker module 302 includes values within the desired range of values over a period of time, the notification module 304 may trigger the recovery threshold. In examples, the notifications may be directly provided to users. In examples, the notifications may be accessed by multiple different caregivers to synchronize their handling of the patient, e.g., if the different caregivers are monitoring patient's post-surgery. The notification alert may be a unique notification tailored for a specific patient. The user behavior module 316 may monitor certain behaviors that lead to biomarker data being within the desired range, which may help users maintain good health outcomes.

In examples, the user behavior module 316 may receive, use, and/or analyze, consumer behavior data and/or population health data. Population health data may be used to determine normative behavior. The normative behavior may allow the user behavior module 316 to determine how a user's health compares to others. Population consumer data and/or population health data may be used to identify segments of the population that may be at increased risk because of certain characteristics. In examples, the population health data may be gathered from specific groups such as age, race, geography, fitness levels, and the like.

The consumer data may help indicate certain health risks and how to improve certain behaviors. For example, the user behavior module 315 may determine that the individuals that purchase a lot of high salt foods, processed foods, snacks that may cause water retention, and the like, may be at risk of disease. By analyzing a user's food purchases, user behavior module 315 may be able to identify how to help the user improve their diet. The system may determine a recommended action for the user based on a biomarker and the consumer data (e.g., the consumer behavioral data of the user and/or the population consumer data).

The self-care/health management module 318 may provide suggestions or recommendations to users to manage their health issues. In examples, the self-care/health management module 318 may provide a dashboard with daily recommendations to encourage them to practice good health, for example, decrease their caffeine consumption increase their water intake, get some physical activity, stress management, meditation, etc. As such, the self-care/health management module 318 may help users to effectively manage their conditions. In examples, the dashboard may be provided directly to users. In examples, the dashboard may be provided to healthcare professionals to help monitor and manage treatment recommendations for patients.

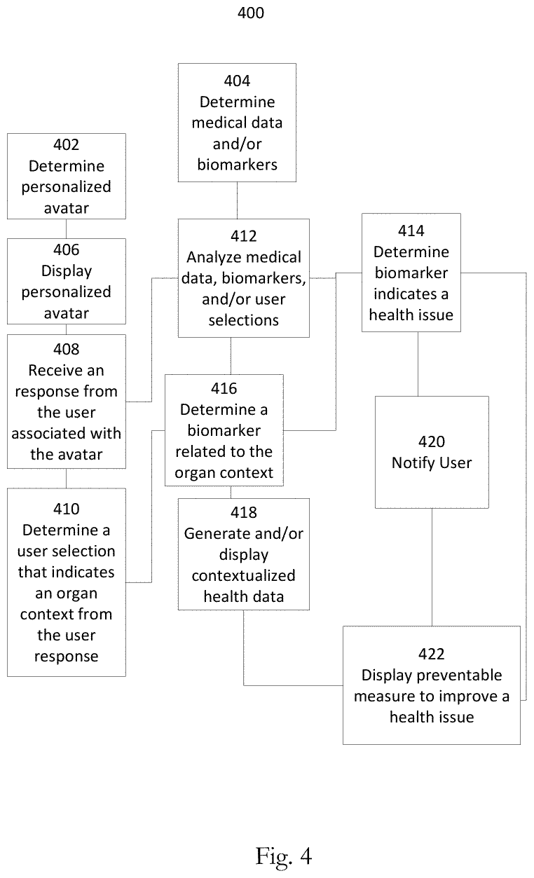

FIG. 4 depicts an example method 400 for providing personalized medical data, statuses, and/or recommendations. At 402, a personalized avatar may be determined. The personalized avatar may be unique and personalized for individual using the avatar. At 404, medical data and/or biomarkers may be determined. The medical data and/or biomarkers may be unique and personalized for the individual using the digital avatar. The medical data and/or biomarkers may include errors and/or conflicting data. Errors and/or conflicting data may be resolved as disclosed herein. For example, the conflicting data may be detected, may be analyzed, and may be resolved such that data is consistent with historical data.

At 406, the personalized avatar determined at 402 may be displayed to the individual using the avatar. At 408, the personalized avatar may receive a response from the user associated with the personalized avatar. At 410, based on the response by the user at 408, the personalized avatar may determine a user selection that indicates an organ context from the user response. At 412, based on the response by the user at 408, the medical data, biomarkers, and/or user selections may be analyzed. At 416, based on the medical data, biomarkers, and/or user selections of the user analyzed at 412 and the organ context from the user response at 410, a biomarker related to the organ context may be determined. At 418, based on the biomarker determined related to the organ context at 416, the personal avatar may generate and/or display contextualized health data. At 422, based on the contextualized health data generated at 418, the personalized avatar may display preventative measures to improve health issues. At 414, based on the biomarkers analyzed at 412 and the biomarker determined related to the organ context at 416, the personalized avatar may determine that the biomarker indicates a health issue. At 422, based on the health issue indicated at 414, the personalized avatar may display preventative measures to improve the health issue. At 420, based on the health issue indicated at 414, the user may be notified of the health issue. At 422, after the user is notified of the health issue at 414, the personalized avatar may display preventative measures to improve the health issue.

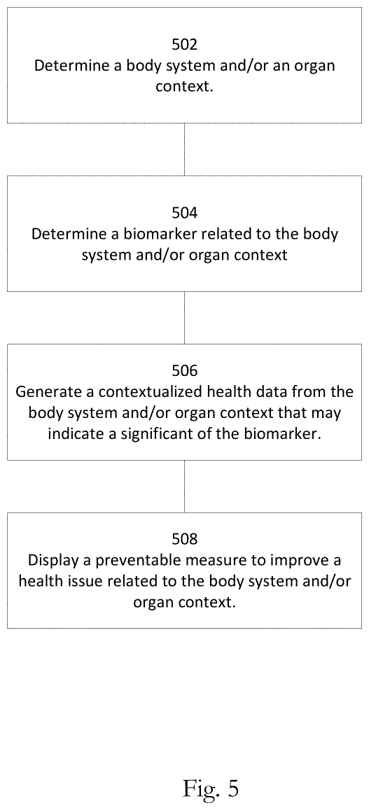

FIG. 5 depicts an example method for using an organ context and/or a biomarker to provide personalized medical data, statuses, and/or recommendations. At 502, a body system and/or an organ context may be determined. The body system and/or organ context may be related to a body system, a group of body systems, a single organ, a group of organs, and organ system, and/or the like. For example, the organ context may be a heart and lungs. As another example, the body system may be the circulatory system.

In an example, an avatar may be displayed to a user and may be associated with a body system and/or an organ context. For example, the avatar may be shown to the user. The avatar may display one or more body systems and/or organs. The body system may be related to one or more body systems of the avatar. The organ context may be related to the one or more organs of the avatar. In an example, it may be determined that the organ context is associated with the heart, and the avatar may be displayed such that the heart of the avatar is highlighted. In an example, it may be determined that the body system is the circulatory system, and the avatar may be displayed such that the circulatory system of the avatar is highlighted. In example, it may be determined that the organ context is the heart, and the body system is the circulatory system, and the avatar may be displayed such that the heart and circulatory system of the avatar are highlighted.

In an example, a user interface may allow the avatar to be customized by the user. For example, a user interface may be provided to the user to allow the user to customize the avatar, which may encourage a user to engage with the app. A customized avatar may allow the user to identify with the avatar such that the user may be concerned about the avatar's well-being as biometric data is displayed with relation to the avatar.

In an example, a body system and/or an organ context may be determined by receiving a selection from the user. The user selection may indicate the organ context. For example, the user may select (e.g., click on) a portion of the avatar, such as the chest of the avatar. Organ systems in the portion of the avatar selected by the user may be displayed to the user to indicate one or more organ contexts. The user may select an organ context from the one or more organ contexts. For example, the user may be presented with a heart, a lung, a liver, intestines, a combination thereof, and/or the like when the user touches the chest of the avatar.

The user selection may indicate the body system. For example, the user may click on a portion of the avatar, such as the chest of the avatar. Body systems associated with the portion of the avatar selected by the user may be displayed to the user to indicate one or more body systems. The user may select a body system from the one or more body systems. For example, the user may be presented with the muscular system, respiratory system, the circulatory system, a combination thereof, and/or the like when the user touches the chest of the avatar.

In an example, a body system and/or an organ context may be associated with a body system, a group of body systems, a group of body systems related to a biomarker, a specific organ, a group of related organs, a group of organs related to a biomarker, a combination thereof, and/or the like. In an example, a group of organs may be related according to a biomarker, such as blood pressure. Blood pressure may be associated with dizziness. Blood pressure may be related to the heart and/or brain. At 504, a biomarker may be determined. For example, a biomarker that may be related to the body system and/or the organ context may be determined. The biomarker may be determined by analyzing data associated with the selected body system and/or organ context. The data may be received from one or more sources, such as a database, another device, a sensor, an electronic medical record, and/or the like. In an example, the biomarker may be determined using another device. The device may be, for example, a wearable device, medical device, medical instrument, and/or the like. The device may be any device described herein. For example, the device may be an EKG, an x-ray machine, a glucose monitor, and/or the like.

In an example, the biomarker may be retrieved from a database. The biomarker may be included in medical and/or health data for an individual, such as an electronic medical record. The biomarker may be included in medical and/or health data for a population, such as a group of electronic medical records, a medical study, hospital records, a database for medical research, a database used by one or more wearables, a combination thereof, and/or the like.

At 506, contextualized health data may be generated from the body system and/or organ context. The contextualized health data may indicate a significance of a biomarker. The significance of a biomarker may be determined by comparing the biomarker to a threshold. The significance of a biomarker may be determined by comparing the biomarker against the model associated with a disease. The model may be an artificial intelligence model, such as described herein. The model may be a risk model. For example, the risk model may indicate that a patient has heart disease, and the biomarker may indicate that the patient is at risk for a heart attack. As an example, the patient may be obese, and the biomarker may indicate that their cholesterol is high.

In an example, the biomarker may be displayed within a range to indicate a significance of the biomarker. The range may show what may be considered normal and/or healthy. The range may indicate what may be considered abnormal and/or unhealthy.

In an example, the biomarker may be displayed with an indication of a likelihood of an outcome to indicate a significance of the biomarker. The biomarker may indicate a likelihood of a negative outcome. For example, the biomarker may indicate that a user is 50% more likely to develop heart disease.

At 508, a preventative measure may be displayed. The preventative display may improve a health issue related to the body system and/or the organ context. An action may be displayed that may assist in moving the biomarker below a threshold. For example, the preventative measure may indicate that a patient should try medication, reduce coffee, and/or contact the doctor to reduce blood pressure. An action may be displayed that may assist in improving and overall health of a patient. For example, and action may indicate that a patient may lose weight to improve their overall health. An action may be displayed that may be based on the body system and/or the organ context. For example, if the user has selected lungs as the organ context, the program may suggest an action to improve lung health. The preventative measure may be determined based on a biomarker, contextual data, organ contacts, and/or the like.

In an example, the biomarker may be displayed to the user. The biomarker may indicate the status of a measurement, such as a current heart rate. The biomarker may indicate a trend, such as a heart rate variability (HRV) over a time period (e.g., a week).

In an example, data associated with a user selection, an organ context, and/or the biomarker may be tracked (may be continued to be tracked). For example, it may be determined how many times a user looks at their heart rate, indicates a headache, and/or the like. This tracked data may be used to determine a focus for the data that may be presented to the user such that the data may be relevant to a user's health concerns.

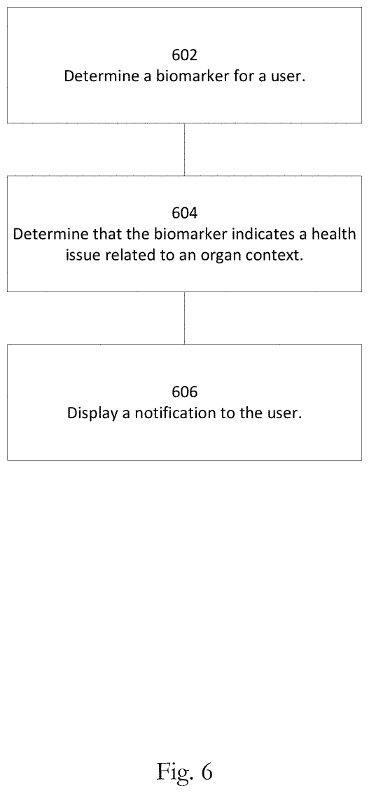

In an example, an indication may be received that may confirm a health issue related to an organ context. For example, it may be determined from an electronic medical record that a doctor has confirmed that a patient has a heart issue. FIG. 6 depicts an example method for using an organ context and/or a contextual health data to provide a personalized medical data notification. At 602, a biomarker may be determined for a user. The biomarker may be determined using any of the methods described herein. For example, a biomarker (that may be related to an organ context) may be determined. The biomarker may be determined by analyzing data associated with the selected organ context. The data may be received from one or more sources, such as a database, another device, a sensor, an electronic medical record, and/or the like. In an example, the biomarker may be determined using another device. The device may be, for example, a wearable device, medical device, medical instrument, and or the like. The device may be any device described herein. For example, the device may be an EKG, an x-ray machine, a glucose monitor, and/or the like.

In an example, the biomarker may be retrieved from a database. The biomarker may be included in medical and/or health data for an individual, such as an electronic medical record. The biomarker may be included in medical and/or health data for a population, such as a group of electronic medical records, a medical study, hospital records, a database for medical research, a database used by one or more wearables, a combination thereof, and/or the like.

At 604, it may be determined that the biomarker may indicate a health issue related to a body system and/or an organ context. For example, the biomarker may be compared to a threshold. The threshold may be associated with a health issue. The threshold may indicate that a health issue may be present when a biomarker exceeds the threshold. In an example, an issue related to heart health may be determined when a biomarker (e.g., a cholesterol level) exceeds a threshold (e.g., a cholesterol threshold). In an example, a health issue related to diabetes may be determined when a biomarker (e.g., a glucose level) exceeds a threshold (e.g., a glucose threshold). It may be determined that the biomarker indicates a health issue related to a body system and/or an organ context when the biomarker is compared against the model associated with a disease. The model may be an artificial intelligence model, such as a pre-trained neural network, and/or a risk model, such as a medical study that indicates that patients with heart disease may be at risk for a heart attack when a cholesterol level is over a threshold value.

It may be determined at the biomarker indicates a health issue related to a body system and/or an organ context (e.g., by using a biomarker that may be received and/or determined using another device). For example, the device may be a wearable device, medical device, a medical instrument, and/or the like. Further examples for determining a biomarker are described herein. At 606, a notification may be displayed to the user. The notification may include contextualized health data, a biomarker, an organ context, a health issue, a combination thereof, and/or the like. The notification may involve an avatar. For example, a health issue may be displayed using the avatar. In an example, the chest of the avatar may be highlighted to indicate an issue with an organ in the chest region. A user may click the region, and the display may focus on the heart to indicate that there is an elevated heart rate, high cholesterol level, elevated blood pressure level, and/or the like.

In an example, a significance of the biomarker may be determined. The significance of the biomarker may be determined by comparing the biomarker to a threshold. The significance of a biomarker may be determined by comparing the biomarker against the model associated with a disease. The model may be an artificial intelligence model, such as described herein. The model may be a risk model. For example, the risk model may indicate that a patient has heart disease, and that the biomarker may indicate that the patient is at risk for a heart attack. As an example, the patient may be obese, and the biomarker may indicate that their cholesterol is high.

In an example, the biomarker may be displayed within a range to indicate a significance of the biomarker. The range may show what may be considered normal and/or healthy. The range may indicate what may be considered abnormal and/or unhealthy.

In an example, the biomarker may be displayed with an indication of a likelihood of an outcome to indicate a significance of the biomarker. The biomarker may indicate a likelihood of a negative outcome. For example, the biomarker may indicate that a user is 50% more likely to develop heart disease.

In an example, data associated with a user selection, an organ context, and/or the biomarker may be tracked (may be continued to be tracked). For example, it may be determined how many times a user looks at their heart rate, indicates a headache, and/or the like. This tracked data may be used to determine a focus for the data that may be presented to the user such that the data may be relevant to a user's health concerns.

In an example, an indication may be received that may confirm a health issue related to an organ context. For example, it may be determined from an electronic medical record that a doctor has confirmed that a patient has a heart issue.

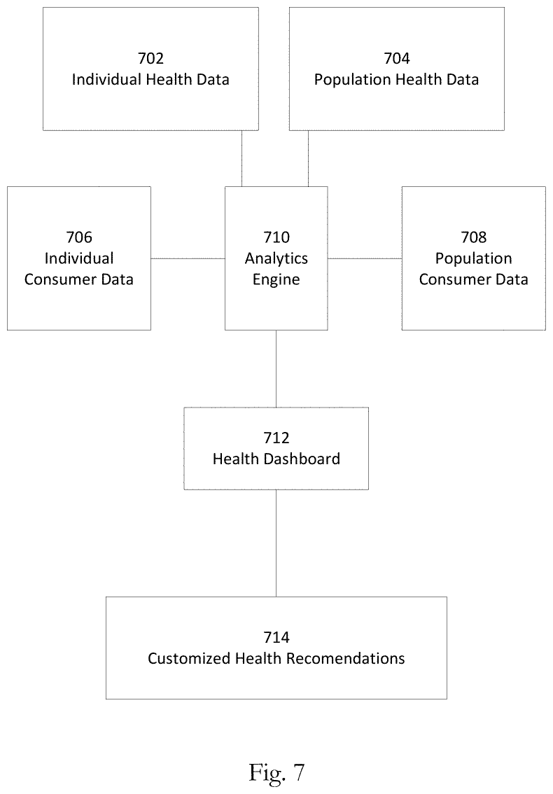

FIG. 7 depicts an example block diagram of an example system that may include one or more devices to provide a customized health recommendation. Example systems described herein may receive data that may be analyzed to provide the customized health recommendation. The data may include health data. The health data received may be individual health data 702 or population health data 704. The individual health data 702, may include biometrics and/or test results for a user.

The population health data 704 may include data related to health for a population. The population health data 704 may be used to determine normative behavior. The population health data 704 may include normative behavior. The normative behavior may be used to evaluate how the population health data 704 may affect an individual. For example, a normative behavior determined from population health data 704 may indicate that individuals that are sedentary may be at risk of obesity.

The population health data 704 may include data that may identify segments of the population that may be at increased risk because of certain characteristics. In examples, the population health data 704 may be gathered from demographic groups such as age, race, geography, fitness levels, a combination thereof, and/or the like. The population health data 704 may indicate factors that provide normative feedback and/or identify who belong to a population at risk of disease.

Population consumer data 708 may be determined from social media platforms. In examples, users may view and/or click on a health-related video, an ad, or a post by somebody. There may be analytics tabulating the number of views and/or clicks. Sometime later (e.g., in the next several days or hours), the social media platforms may present similar videos, similar products, similar recommendations, and similar posts to individuals with similar health-related issues based on the number of views and/or clicks. The social media platforms may make assumptions, predictions, and/or hypotheses about why the individuals interacted with the health-related data the individuals are viewing or clicking. In examples, if somebody has knee pain, they may be looking on sites for sleeves that they can wear on their knees (e.g., to help them with arthritis or knee pain). The views and clicks on those sites may trigger similar recommendations or websites related to pain medication, physical therapy, doing certain exercises, or to diet and fluid retention, etc.

Individual consumer data 706 may include data regarding purchases made by an individual, purchasing behavior by an individual, financial decisions made by an individual, information regarding financial accounts, and the like. In examples, there may be situations that individual consumer data 706 may help to confirm certain health risks to the individuals. For example, there may be an indicator in the individual health data 702 that indicates that the individual may be at risk for certain health issues. The individual consumer data 706 may then be evaluated to confirm that the health issues exist. In examples, in cases of hypertension, if someone thinks they may be at risk for hypertension, the individual health data 702 may start to show that indicator. In examples, the system may monitor (e.g., watch) the consumer patterns of individuals at risk for hypertension. If the individuals show an interest in consumer items related to hypertension (e.g., food items that may increase the individual's likelihood of developing hypertension), monitoring such patterns may help confirm the individuals are at risk for hypertension. In examples, there may be data discrepancies in the analytics, which may be confirmed in a conflict resolution module (e.g., such as analytics engine 710).

In examples, the individual consumer data 706 may help indicate certain health risks and how to improve certain behaviors. For example, individuals that purchase a lot of high salt foods, processed foods, snacks that that have risk for water retention, etc., may indicate that their health behaviors related to their nutrition are potentially contributing to the risk factors. By the individual's food purchases, it may be possible to identify how to help them improve their diet. For example, if individuals are buying cigarettes and decongestants that raise blood pressure, those items may be considered in the equation to help determine the risk profile and what may be causing an underlying spike that the individuals may see on a wearable related to their blood pressure.

Analytics engine 710 may analyze, modify, use, and/or create data from individual health data 702, population health data 704, individual consumer data 706, and/or population consumer data 708. In an example, analytics engine 710 may integrate the individual health data 702 and the population health data 704. Integrating the individual health data 704 and the population health data 704 may allow individuals may evaluate their own health and may allow individuals to determine how their health compares to others. In examples, individuals may compare individual health risks to population health risk.

In an example, analytics engine 710 may integrate the individual health data 702 and the population health data 704 to enhance consumer experiences. For example, a healthcare organization may use the integrated data to help evaluate healthcare products, have social media perspectives, and/or develop different retail perspectives. In examples, organizations such as grocery stores may compile population health data 704 based on consumer data and purchasing behavior that go into analytics engines. The analytics engine 710 may lead to population-based promotion and outreach, and also to individual-level outreach. In examples, at a consumer level, individuals may purchase products that relate to sleep. The population health data 704 may suggest patterns in individuals having problems with sleep. The individual health data 702 may be calculated from sleep data (e.g., from a Fitbit or an Apple watch), data about the individual's fluid intake, and/or data about one or more of the individual's medications. The individual health data 702 may be combined with the patterns found in the population health data 704. From there, the combination of data sources may identify and predict people have problems with stress, sleep (e.g., lack of sleep), and/or pain, etc.

In examples, individual health data 702 and population health data 704 may be input into the analytics engine 710 and analyzed by the analytics engine 710. The analytics engine 710 may perform the analysis using normative data and may output results to a health dashboard 712. The health dashboard 712 may present customized health recommendations 714. In examples, the individual customer data 706 and the population customer data 708 may be inputted (e.g., in addition to or separate from the individual health data 702 and/or population health data 704) and analyzed by the analytics engine 710. Examples of the individual customer data 706 and the population consumer data 708 may consider (e.g., pull in) consumer data from social media platforms. Consumer data from social media platforms may include where users are searching online for information (e.g., about stress, anxiety, or depression), if users have purchased medication (e.g., sleep medication) over the counter, if users have dramatically increased or decreased the number of social media posts that they have made, or any other indicators of a brewing depression or challenges that could contribute to depression.

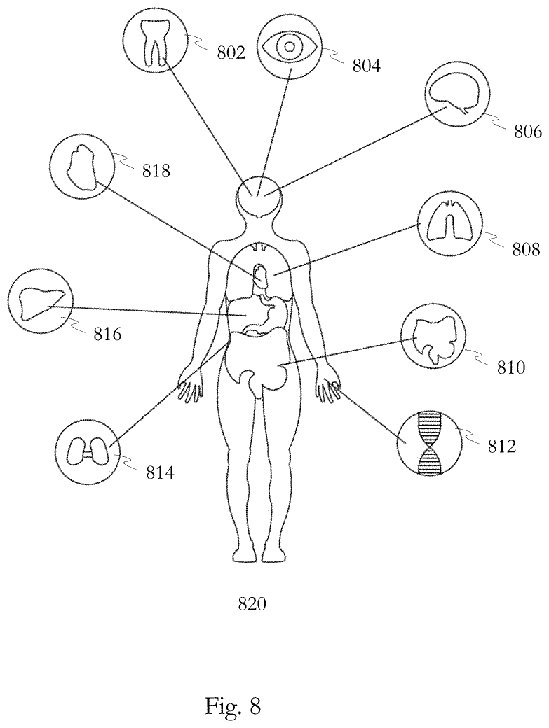

FIG. 8 depicts an example user interface that may include a customizable avatar 820 for providing personalized medical data. Avatar 820 maybe customizable by a user. For example, a user may customize avatar 820 such that the avatar 820 may reflect the user. The user may change the height, weight, skin color, and other features of the avatar 820. By customizing the avatar 820, the user may be more inclined to interact with the avatar 820.

The avatar 820 may include one or more body systems and/or organ contexts. The body systems and/or organ contexts may be used by a user to indicate areas of concern for the user. The body systems and/or organ contexts may be used by a program to indicate areas of concern for the user. In an example, the user may select a portion of the avatar 820 to indicate that the user is experiencing a health-related issue related to a body system or organ context. For example, the user may select the head of the avatar 820 to indicate that the user is experiencing head pain. In an example, a program may indicate that there may be an issue at a portion of the avatar related to the lungs.

The avatar 820 made include a dental context 802, vision context 804, a brain context 806, a lung context 808, a stomach context 810, a blood context 812, a kidney context 814, a liver context 816, and/or a heart context 818. As described herein, other body systems and organ contexts may be included and/or may be displayed using the avatar 820.

The dental context 802 may include information regarding body systems and/or organs related to a mouth of a user. The body systems and/or organs may include teeth, lips, a tongue, and the like. In an example, a user may select dental context 802 to indicate that the user may be experiencing tooth pain. The program may analyze data related to the user, such as biometric data, and may indicate to the user that the user may have a cavity. In an example, a program may analyze biometric data associated with the user and may determine that the user may be dehydrated. The program may use dental context 802 to indicate to the user that there may be an issue and may suggest that the user take some action(s) (e.g., drink water).

The vision context 804 may include information regarding body systems and/or organs related to the vision of a user. The body systems and/or organs may include eyes, optic nerves, bones around the eye sockets, the brain, and/or the like. In an example, a user may select the vision context 804 to indicate that the user is experiencing vision issues. The program may analyze data related to the user, such as biometric data, and may indicate to the user that the user may be experiencing eye fatigue from viewing a computer screen. In an example, the program may analyze biometric data associated with the user and may determine that the user may be experiencing eye pain. The program may use the vision context 802 to indicate to the user that there may be an issue and may suggest that the user see an eye doctor.

The brain context 806 may include information regarding body systems and/or organs related to the cognitive function of a user. Such body systems and/or organs may include nerves, the skull, the brain, and/or the like. In an example, a user may select the brain context 806 to indicate that the user may be experiencing head pain. The program may analyze data related to the user, such as biometric data, and may indicate to the user that the user may be experiencing a headache. In an example, the program may analyze data related to the user, such as biometric data, and may indicate to the user that the user may be experiencing stress. The program may use brain context 806 to indicate to the user that there may be an issue and may suggest that the user try a breathing exercise.

The lung context 808 may include information regarding body systems and/or organs related to the respiratory system of a user. Such body systems and/or organs may include the lungs, the heart, the diaphragm, and/or the like. In an example, a user may select the lung context 808 to indicate that the user may be experiencing shortness of breath. The program may analyze data related to the user, such as biometric data, and may indicate to the user that the user may be experiencing asthma. In an example, the program may analyze data related to the user, such as biometric data, and may indicate to the user that the user may have missed a dose of asthma medication. The program may use the lung context 808 to indicate to the user that there may be an issue and may suggest that the user take a dosage of asthma medication.

The stomach context 810 may include information regarding body systems and/or organs related to the digestive system of a user. Such body systems and/or organs may include the intestines, the blood, the stomach, and/or the like. In an example, a user may select the stomach context 810 to indicate that the user may be experiencing abdominal pain. The program may analyze data related to the user, such as biometric data, and may indicate to the user that the user may be experiencing heartburn. In an example, the program may analyze data related to the user, such as biometric data, and may determine that the user may benefit from a dose of insulin. The program may use the stomach context 810 to indicate to the user that there may be an issue and may suggest that the user take insulin.

The blood context 812 may include information regarding body systems and/or organs related to the blood of a user. Such body systems and/or organs may include the blood, the heart, bone marrow, and/or the like. In an example, a user may select the blood context 812 to explore the results of a DNA sequencing that was performed for the user. The program may analyze the DNA sequencing, may determine that the user may be at risk for heart disease, and may notify the user of the risk for heart disease. In an example, the program may analyze data related to the user, such as biometric data, and may determine that the user may have high cholesterol. The program may use the blood context 812 to indicate to the user that there may be an issue and may suggest that the user schedule a visit with a doctor.

The kidney context 814 may include information regarding body systems and/or organs related to the urinary system of a user. Such body systems and/or organs may include the blood, the kidneys, the bladder, and/or the like. In an example, a user may select the kidney context 814 to indicate that the user is experiencing kidney pain. The program may analyze one or more biometrics related to the user, may determine that the user is at risk for kidney stones, and may notify the user of the risk for kidney stones. In an example, the program may analyze data related to the user, such as biometric data, and may determine that the user may be at risk for a urinary tract infection. The program may use the kidney context 814 to indicate to the user that there may be an issue and may suggest that the user schedule a visit with a doctor.

The liver context 816 may include information regarding body systems and/or organs related to the excretory system of a user. Such body systems and/or organs may include the blood, the liver, the gallbladder, and/or the like. In an example, a user may select the liver context 816 to indicate that the user is experiencing abdominal pain. The program may analyze one or more biometrics related to the user, may determine that the user is at risk for hepatic encephalopathy, and may notify the user of the risk for hepatic encephalopathy. In an example, the program may analyze data related to the user, such as biometric data, and may determine that the user may improve their liver function by avoiding fatty foods. The program may use the kidney context 816 to indicate to the user that there may be an issue and may suggest that the user avoid fatty foods.

The heart context 818 may include information regarding body systems and/or organs related to the heart of a user. Such body systems and/or organs may include the blood, the brain, the heart, and/or the like. In an example, a user may select the heart context 818 to indicate that the user is experiencing chest pain. The program may analyze one or more biometrics related to the user, may determine that the user is at risk for a heart attack, and may notify the user of the risk for heart attack. In an example, the program may analyze data related to the user, such as biometric data, and may determine that the user may improve their heart function by exercising. The program may use the heart context 818 to indicate to the user that there may be an issue and may suggest that the user exercise.

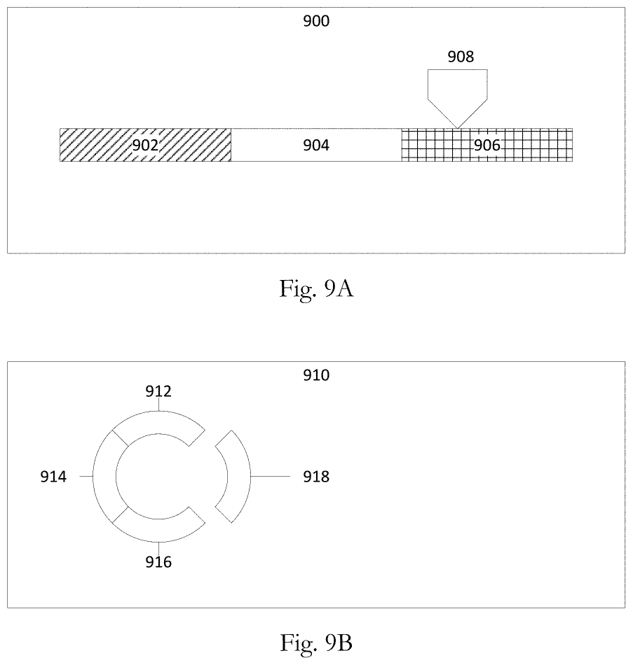

FIG. 9A-B depict example user interfaces for providing personalized medical data, statuses, and/or recommendations. FIG. 9A shows an example interface 900. Interface 900 may provide personalized medical data to a user by showing a test score for the user in comparison to normalized scores for a population and/or in comparison to a range of risk for a disease. For example, the results of a cholesterol test for a user may be shown by displaying the cholesterol score for the user along with the range of scores that may indicate a range of risk for heart disease. At 908, the cholesterol score for the user may be shown. The range of cholesterol scores may be shown using a first range at 906, a second range at 904, and a third range at 902. The range of cholesterol scores may be associated with a good score, an acceptable score, and an at risk score, such that a good range may be shown at 902, an acceptable range may be shown at 904, and an at risk range may be shown at 906. As reflected at 908, the user may have a cholesterol score that is within the at risk range shown at 906. The interface 900 may indicate to the user that the cholesterol score for the user is within an at risk range and that the user may be at risk of heart disease. The range at 906, 904, and/or 902 may display a color, an image, a pattern, and/or the like to indicate a significance. In an example, the location of the range at 902, 904, and/or 906 may indicate a significance (e.g., the left-most range indicating a good range, and the right-most range indicating an at risk range).

FIG. 9B shows an example interface 910. Interface 910 may provide personalized medical data to a user by showing a test score for a user in comparison to normalized scores for a population and/or in comparison to a range of risk for a disease. For example, the results of a cholesterol test for a user may be shown by displaying the cholesterol score for the user along with how the user compares to a population. The scores for the population may be divided into four portions, the first portion at 912, the second portion at 914, the third portion at 916, and the fourth portion at 918. The portions may reflect a level of risk for heart disease. For example, the first portion at 912 may be at the lowest risk for heart disease, the second portion at 914 may be at an acceptable risk for heart disease, the third portion at 916 may be at an elevated risk for heart disease, and the fourth portion at 918 may be at a high risk for heart disease. The score for the user may be shown at 918, which may correlate to a high risk of heart disease. The interface 910 may indicate to the user that the user is at high risk for heart disease and is part of the population that is at high risk for heart disease. The portions at 912, 914, 916, and/or 918 may display a color, an image, a pattern, and/or the like to indicate a significance. In an example, the location of the portion, such as at 912, 914, 916, and/or 918 may indicate a significance.

The interface 900 and/or 910 may include a recommendation that may assist the user in improving their health. For example, the interface 900 and/or 910 may be accompanied by a notification indicating that the user may reduce their cholesterol score by avoiding fatty foods and/or alcohol.

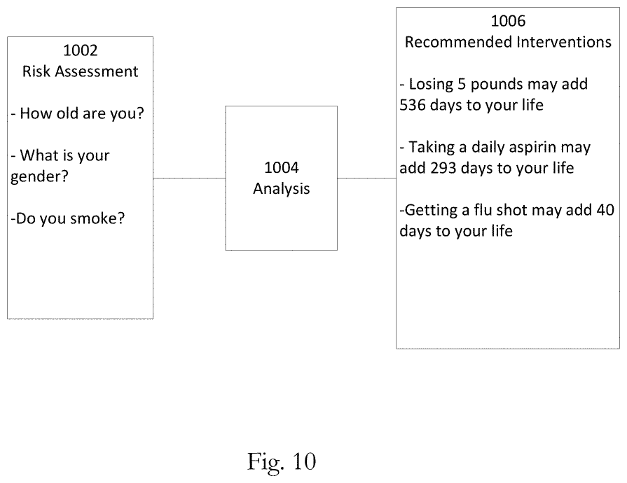

FIG. 10 depicts an example method for providing personalized medical data, statuses, and/or recommendations using risk assessments and/or risk analysis. Risk assessments may be provided at 1002 and may be analyzed at 1004. Based on the analysis at 1004, recommended interventions may be provided at 1006. In examples, risk assessments at 1002 may be based on users providing psychometric or responses to questions. In examples, risk assessments at 1002 (e.g., in addition to or instead of users providing responses to questions), may be based on health data information provided from a wearable, such as a user's current blood pressure, average hours of sleep, medications of the user, or number of steps taken per day by the user. In examples, risk assessments at 1002 (e.g., in addition to or instead of users providing responses to questions and/or receiving wearable), may be based on receiving data of users purchasing certain foods (e.g., high-salt snacks), analyzing those purchases at 1004, and making recommended interventions at 1006 (e.g., cutting down on salt to improve blood pressure).

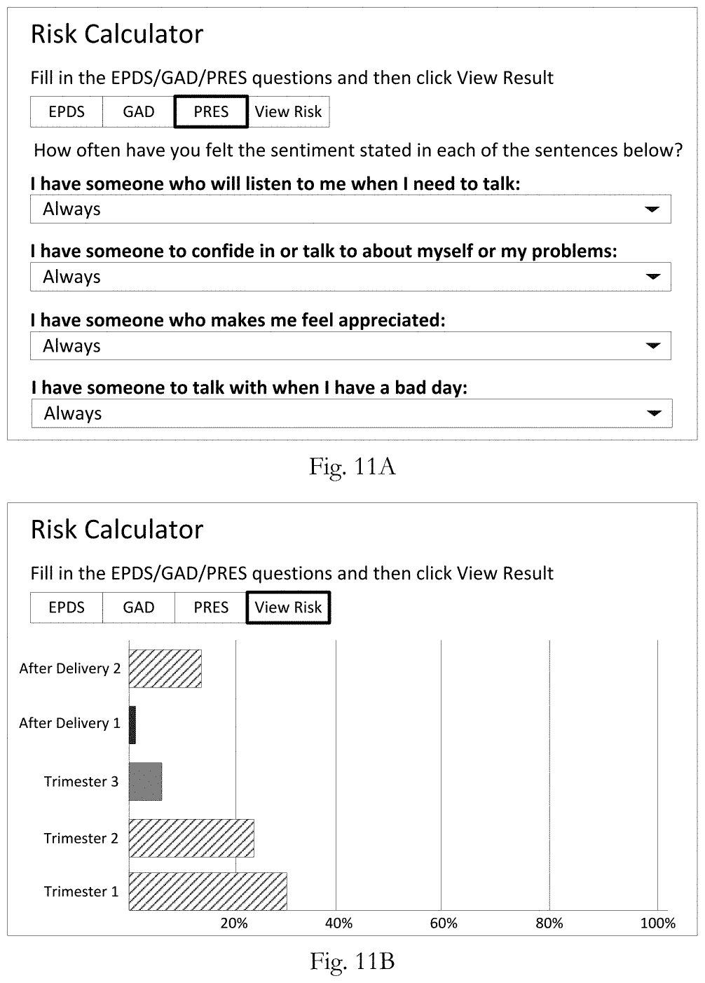

FIG. 11A-B depicts example user interfaces for providing personalized medical data, statuses, and/or recommendations using risk assessments and/or risk analysis. Embodiments disclosed herein may involve performing risk analysis. The risk analysis may determine a probability or a risk of the user experiencing a disease. For example, the risk analysis may determine how likely it may be for a user to be depressed. To perform the risk analysis, embodiments disclosed herein may present the user with a number of questions. For example, a user may be presented with a number of questions to assess the mental state of the user.

FIG. 11A shows an example user interface that may be used to assess a user's risk of depression based on a number of questions. As shown in FIG. 11A, the user may be asked how often they may feel the sentiment stated in questions that are presented. For example, the user may be asked if they have someone who will listen to them when they need to talk. The user's responses may be recorded and may be analyzed. The user's responses may be scored based on the response provided. In an example, if a user responds with “never” to a question, the user may be at a higher risk of depression. In an example, if the user responds with “always” to a question, the user may be at lower risk of depression. The user responses may be scored, and an analysis may be provided to the user.

FIG. 11B shows an example user interface that may be used to provide personalized medical data, risk assessments, and/or recommendations to a user. As show in FIG. 11B, the results of a risk assessment for depression may be presented to a user. The risk assessment may indicate how likely it may be for a user to experience depression. The risk assessment may indicate when it may be likely for the user to experience depression. For example, the risk assessment may indicate that it is more likely that the user will be depressed during a first trimester than a third trimester. The risk assessment may indicate a significance of a risk using a percentage, a graph, an image, a color, a size, and/or the like.

This application may refer to “determining” various pieces of information. Determining the information can include one or more of, for example, estimating the information, calculating the information, predicting the information, or retrieving the information from memory.

Additionally, this application may refer to “receiving” various pieces of information. Receiving is, as with “accessing,” intended to be a broad term. Receiving the information can include one or more of, for example, accessing the information, or retrieving the information (for example, from memory). Further, “receiving” is typically involved, in one way or another, during operations such as, for example, storing the information, processing the information, transmitting the information, moving the information, copying the information, erasing the information, calculating the information, determining the information, predicting the information, or estimating the information.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as is clear to one of ordinary skill in this and related arts, for as many items as are listed.

We describe a number of examples. Features of these examples can be provided alone or in any combination, across various claim categories and types. Further, embodiments can include one or more of the following features, devices, or aspects, alone or in any combination, across various claim categories and types.

1. A device for providing personal medical data, the device comprising:
a processor, the processor configured to:
display a graphic of a human body
receive, from a user, a user input associated with a location on the graphic of a human body;
determine an organ context based on the location on the graphic of the human body;
determine a biomarker related to the organ context;
generate contextualized health data that indicates a significance of the biomarker in relation to the organ context; and
in response to the user input, display the contextualized health data, a recommended action, and an indication of an amount of time that the user's life may be extended by the user performing the recommended action.

2. The device of claim 1, wherein the processor is further configured to:
determine that a value of the biomarker is outside of an acceptable range of values; and
display, in a location associated with the organ context, a notification indicating for the user to review the biomarker, wherein the user input comprises selecting the notification.

3. The device of claim 1, wherein the processor is further configured to determine the recommended action based on the biomarker and consumer data associated with the user.

4. The device of claim 1, wherein the organ context indicates a context of a plurality of organs based on a shared association with a location, a biomarker, or a disease.

5. The device of claim 1, wherein the processor is further configured to prompt the user to answer mental health questions, wherein the user input comprises user responses to the mental health questions.

6. The device of claim 1, wherein the graphic of a human body comprises an avatar that is representative of the user, and wherein the user input comprises the user selecting a body part of the avatar.

7. The device of claim 6, wherein the processor is further configured to display an organ to the user in relation to the avatar, wherein the organ is associated with the organ context.

8. The device of claim 1, wherein the processor is further configured to determine the organ context by determining a user selection that indicates the organ context.

9. The device of claim 1, wherein the processor is further configured to determine the biomarker related to the organ context based on medical data from at least of a wearable device, a medical device, a medical instrument, or a database.

10. The device of claim 1, wherein the processor is further configured to generate the contextualized health data for the organ context that indicates the significance of the biomarker by displaying the biomarker to the user in relation to at least of a range, a threshold, or a risk model.

11. The device of claim 1, wherein the processor is further configured to generate the contextualized health data for the organ context that indicates the significance of the biomarker by:
determining an artificial intelligence model associated with a disease;
determining a probability of the disease using the artificial intelligence model and the biomarker; and
determining the contextualized health data using the probability of the disease, wherein the contextualized data indicates a likelihood of an outcome associated with the disease.

12. The device of claim 1, wherein the processor is further configured to determine the recommended action based on at least one of the biomarker or the contextualized health data, wherein the recommended action indicates an action that can improve a health issue related to the organ context.

13. The device of claim 1, wherein the processor is further configured to determine an indication that confirms a health issue related to the organ context using at least of an electronic medical record, data from a health care professional, a second biomarker, a second contextualized health data, a selection from the user, or an artificial intelligence model.

14. A method for providing personal medical data, the method comprising:
displaying a graphic of a human body
receiving, for a user, a user input associated with a location on the graphic of a human body;
determining an organ context based on the location on the graphic of the human body;
determining a biomarker related to the organ context;
generating contextualized health data that indicates a significance of the biomarker in relation to the organ context; and
in response to the user input, displaying the contextualized health data, a recommended action, and an indication of an amount of time that the user's life may be extended by the user performing the recommended action.

15. The method of claim 14, further comprising
determining that a value of the biomarker is outside of an acceptable range of values; and
displaying, in a location associated with the organ context, a notification indicating for the user to review the biomarker, wherein the user input comprises selecting the notification.

16. The method of claim 14, further comprising determining the recommended action based on the biomarker and consumer data associated with the user.

17. The method of claim 14, wherein the organ context indicates a context of a plurality of organs based on a shared association with a location, a biomarker, or a disease.

18. The method of claim 14, further comprising prompting the user to answer mental health questions, wherein the user input comprises user responses to the mental health questions.

19. The method of claim 14, wherein the graphic of a human body comprises an avatar that is representative of the user, and wherein the user input comprises the user selecting a body part of the avatar.

20. A device for providing a personalized medical data notification, the device comprising
a processor, the processor configured to:
determine a biomarker for a user;
determine an organ context related to the biomarker;
determine that a value of the biomarker is outside of an acceptable range of values; and
generate contextualized health data that indicates a significance of the biomarker in relation to the organ context; and
display, in a location associated with the organ context, a notification indicating for the user to review the biomarker;
receive user input, wherein the user input comprises selecting the notification; and
in response to the user input, display the contextualized health data and a recommended action.
